(19) United States
(12) Patent Application Publication
(10) Pub. No.: US 2026/0185033 A1
(43) Pub. Date: Jul. 2, 2026

(54) COMPLETELY NONINVASIVE MULTI-ANALYTE MONITORING SYSTEM FOR CELL CULTURE PROCESSES

(71) Applicant: UNIVERSITY OF MARYLAND, BALTIMORE COUNTY, Baltimore, MD (US)

(72) Inventors: Govind RAO, Ellicott City, MD (US); Michael TOLOSA, Columbia, MD (US); Vida RAHMATNEJAD, Providence, RI (US); Vikash KUMAR, Catonsville, MD (US); Xudong GE, Woodstock, MD (US)

(21) Appl. No.: 19/549,491

(22) Filed: Feb. 25, 2026

Related U.S. Application Data
(63) Continuation of application No. PCT/US24/44988, filed on Sep. 3, 2024.
(60) Provisional application No. 63/536,090, filed on Sep. 1, 2023.
(60) Provisional application No. 63/685,103, filed on Aug. 20, 2024.

(51) Int. Cl. C12M 1/34; C12M 1/00

(52) U.S. Cl. CPC C12M 41/32; C12M 23/22; C12M 29/04; C12M 31/02; C12M 41/34

(57) ABSTRACT

A noninvasive system and method for simultaneous monitoring of species in a cell culture medium is described. Instead of direct contact with the culture media, the measurements can be made through permeable membranes via either a port in the culture vessel wall or a port in a flow cell. The noninvasive monitoring system and method can offer accurate, and contamination-minimized monitoring of critical process parameters including dissolved O₂, pH, and dissolved CO₂. These advancements will enhance the control and optimization of cell culture processes, promising improved cell culture performance.

### CROSS REFERENCE TO RELATED APPLICATIONS

This application claims priority to U.S. Provisional Patent Application No. 63/536,090 filed on Sep. 1, 2023, in the name of Govind RAO et al. entitled “NON-INVASIVE OXYGEN AND CARBON DIOXIDE MONITORING SYSTEM FOR BIOREACTORS,” and U.S. Provisional Patent Application No. 63/685,103 filed on Aug. 20, 2024 in the name of Govind RAO et al. entitled “COMPLETELY NONINVASIVE MULTI-ANALYTE MONITORING SYSTEM FOR CELL CULTURE PROCESSES,” both of which are hereby incorporated by reference herein in their entirety.

### FIELD OF THE INVENTION

A noninvasive system and method for simultaneously monitoring species including, but not limited to, dissolved O₂, pH, glucose, and dissolved CO₂, in a fluid, e.g., a cell culture medium, is described. Instead of direct contact with the fluid, the measurements are made through permeable membranes via either a port in a container wall or a port in a flow cell.

### BACKGROUND OF THE INVENTION

Cell culture is a common practice in academia and the pharmaceutical industry and is conducted for different purposes, such as investigating the physiology or biochemistry of cells, studying the effect of drugs or chemicals on cells, fabricating artificial tissues, and manufacturing biologics. Any changes in the environmental condition of cells can affect the cell function. For example, it is known that the pH level in the cell culture medium directly impacts the enzymatic activity and metabolism of cells. Normal cells achieve optimal growth within an alkaline pH range, while cancer cells tolerate a wider pH range, including acidic environments. Therefore, maintaining an optimal pH specific to the cell culture process is crucial. Dissolved gases also impact cellular physiology. For example, a high partial pressure of CO₂ reduces pH, which affects cell metabolism and alters protein properties. Similarly, low partial pressures of CO₂ negatively affect cell growth. Furthermore, studies have shown that hypoxic conditions (less than 10% O₂) promote stem cell differentiation.

Considering the importance of 02, CO₂, and pH in cell behavior, these analytes are often monitored throughout the cell culture process. The data obtained from sensors not only provides a thorough understanding of the cell culture environment but can also be utilized to develop control systems for maintaining a desirable level of critical process parameters. Furthermore, to ensure compliance, the Food and Drug Administration (FDA) encourages the use of process analytical technologies (PATs) in the biopharmaceutical industry through guidance on “Innovative Pharmaceutical Development, Manufacturing, and Quality Assurance.” This has led to the development of various sensors for cell culture processes (Abou-el-Enein et al. 2021; Klein et al. 2021; Lashkari 2017; Rao. 2020).

Electrochemical sensors offer robust and efficient performance and are the most commonly used sensors for monitoring DO and pH. However, their bulkiness makes them less appropriate for small-scale cell culture processes. On the other hand, optical sensors are small and ideal for low-volume cultures. Their minimally invasive nature reduces the chance of contamination. However, they still require direct contact with the cell culture environment to conduct the measurements. Single on-chip sensors and sensing cell culture flask (SCCF) sensors are newly developed techniques for monitoring pH and DO. In another technique, a Clark-type DO sensor is coupled with a BLE chipset (a microcontroller used for data processing and transmission) wherein the chip is embedded in the bottom of the vessel and in contact with the cell culture. Furthermore, Wavepod™ II-pHOPT from GE™ Healthcare, iTube pH Bioreactor from PreSens®, TurFluor pH from Fitnesse, and OptiSens pH from Sartorius® are among the commercially available optical sensors. An optical sensor consisting of a sensor cassette, a pump, and a flow-through cuvette was recently reported (Kattipparambil Rajan et al. 2016). In this system, the sample is transferred to a light emitting diode (LED) cassette and the pH determined using colorimetric indicator pH analysis based on absorption of light at about 545 nm and about 680 nm. No contact with the cell culture environment is required. The measurements via this method have high accuracy and sensitivity but, as admitted by the investigators, errors could result from indicator-protein binding (e.g., the phenol red indicator present in most standard stem cell culture media) or turbidity from contamination.

Electrochemical and optical sensors are commonly utilized as monitoring systems for monitoring DCO₂ throughout the cell culture process. Off-gas analyzers offer an alternative technology for monitoring DCO₂ without direct contact with the cell culture medium (Kroll et al. 2019). This technique is inexpensive and highly stable however, it does not provide real-time DCO₂ values in the media. Another method for DCO₂ monitoring is the circulation direct monitoring and sampling system (CDMSS). The technique allows sampling without interrupting culture agitation and can measure CO₂ in gas and liquid phases. However, CDMSS requires a system to prevent bypass component clogging and is not appropriate for small volumes of cultures (Takahashi et al. 2017).

Microfluidic systems are another type of technology developed for monitoring different analytes in bioreactors. In these techniques, the sample is transferred to the sensor for measurements. Some examples of this type of technology are biophotonic lab-on-a-chip for pH monitoring, multi-sensor microsystem for monitoring pH and DO, magnetic optical sensor particles (MOSePs) and Chip-based monitoring system designed for monitoring DO. Hydrogel microarray sensor has been reported for monitoring DO and pH via optical sensors positioned externally to the bioreactor (Lee et al. 2008). This technique offers reliable measurements however, prior to the disclosure herein, the sensing part has come in direct contact with the cell culture medium, which has the potential to impact/contaminate the cell culture medium. In another example, flow loop developed by SBI is a commercially available technology for monitoring DO and pH throughout the process. This method enables the monitoring of DO and pH from outside of the vessel and can be adjusted for various types of vessels. However, one drawback of this technology is that the luminescent dye is in direct contact with the cell culture medium throughout the process, which raises concerns regarding the cytotoxicity of the dye. In general, all the aforementioned methods are invasive and limited to monitoring one or two critical analytes and require the integration of the sensing components with the cell culture vessel.

From the above discussions, it can be seen that nearly all existing technologies require some level of contact with the cell culture environment, i.e., are invasive, posing a risk for contamination or interferences. Accordingly, there continues to be a need for completely noninvasive “non-contact” sensing technology that is capable of online monitoring of DO, pH, and DCO₂ while addressing the limitations of the prior art technologies.

### SUMMARY OF THE INVENTION

In some aspects, a system for noninvasively detecting and quantitating species within a container is described, said system comprising:

the container, wherein the container comprises at least one hole or port in a wall of the container, wherein a membrane covers, or is positioned within, each hole or port, and wherein the membrane permits the passage of the species to be detected and quantitated therethrough;

at least one sensing device, wherein the at least one sensing device is positioned on, or in proximity to, the membrane, wherein the at least one sensing device is not in contact with any fluid present in the container, and wherein the at least one sensing device collects the species that pass through the membrane, reacts with the species that pass through the membrane, or both; and a detection device for quantitating the species collected in, reacted with, or both collected in and reacted with, the at least one sensing device.

In some other aspects, a flow cell for noninvasively detecting and quantitating species is described, said flow cell comprising:
a layer for positioning of a first sensing device;
a first membrane layer, wherein the first membrane layer permits the passage of a species to be detected by the first sensing device;
a layer comprising a media channel;
a second membrane layer, wherein the second membrane layer permits the passage of a species to be detected by a second sensing device; and
a layer for positioning of the second sensing device,
   wherein the layer comprising the media channel can be communicatively connected to a vessel comprising a medium, wherein the medium comprises species to be detected and quantitated.

In another aspect, a system for noninvasively detecting and quantitating species in a medium contained in a vessel is described, said system comprising:
a flow cell;
a pump; and
a vessel,
   wherein the flow cell comprises:
a layer for positioning of a first sensing device;
a first membrane layer, wherein the first membrane layer permits the passage of a species to be detected by the first sensing device;
a layer comprising a media channel;
a second membrane layer, wherein the second membrane layer permits the passage of a species to be detected by a second sensing device; and
a layer for positioning of the second sensing device,
   wherein the layer comprising the media channel can be communicatively connected to the vessel comprising the medium for detection and quantitation of a species contained therein.

In still other aspects, a system for noninvasively detecting and quantitating species in a medium contained in a vessel is described, said system comprising:
a flow cell;
a pump; and
a vessel,
   wherein the flow cell is communicatively connected to the vessel comprising the medium for detection and quantitation of a species contained therein, and wherein the flow cell comprises:
a flow cell container, wherein the flow cell container comprises at least one hole or port in a wall of the flow cell container, an inlet and an outlet, wherein a membrane covers, or is positioned within, each hole or port, and wherein the membrane permits the passage of the species to be detected and quantitated therethrough;
at least one sensing device, wherein the at least one sensing device is positioned on, or in proximity to, the membrane, wherein the at least one sensing device collects the species that pass through the membrane, reacts with the species that pass through the membrane, or both; and
a detection device for quantitating the species collected in, reacted with, or both collected in and reacted with, the at least one sensing device.

Other aspects and advantages will be more fully apparent from the ensuing disclosure and appended claims.

### BRIEF DESCRIPTION OF THE FIGURES

FIG. 1A. T-flask setup for noninvasive monitoring of pH. Throughout the process, protons diffuse through the cellulose membrane, contact the sensing patch, and measurements are conducted based on the optical sensing technique.

FIG. 1B. An embodiment of a bioreactor already having a permeable bottom for DO and DCO₂ measurements. A gas sampler, as described herein, attaches to the bottom of the bioreactor creating a closed seal with the permeable bottom. O₂ and CO₂ diffuses through the permeable bottom membrane into the gas sampler and flows to a sensor where measurements take place. When not measuring, a desired gas mix (02 or CO₂ enriched) can flow through the sampler to oxygenate or maintain the buffering capacity of the medium in the bioreactor.

FIG. 1C. An embodiment of a shake flask wherein a portion of the flask comprises a gas permeable membrane for DO and DCO₂ measurements. A gas sampler, as described herein, can be positioned to the outside of the shake flask, creating a closed seal with the membrane. 02 and CO₂ diffuses through the permeable membrane into the gas sampler and flows to a sensor where measurements take place.

FIG. 2A. A top view of a sampler attached to a sensor via tubing.

FIG. 2B. A side view of the sampler as well as a representation of the positioning of the sampler relative to the hole of the container or flow cell.

FIG. 2C. An embodiment of a T-flask with a ring comprising the membrane and threaded holes for more reliable and consistent placement of a sampler.

FIG. 3A. Flow cell setup. The sample is drawn from the bioreactor and transferred to the flow cell to conduct simultaneous measurements of dissolved O₂ (DO), pH, and dissolved CO₂ (DCO₂).

FIG. 3B. An embodiment of a flow cell having three holes having a permeable membrane covering, wherein the DCO₂ analysis includes a sampler and tubing for measurement at a sensor (not shown), and the pH and DO analysis includes a sampler with optical sensing patches specific to the species. The DO, pH, and DCO₂ measurements are conducted simultaneously as the sample passes through the flow cell.

FIG. 3C. An embodiment of a flow cell having four holes having a permeable membrane covering, wherein the DCO₂ analysis includes a sampler and tubing for measurement at a sensor (not shown), the glucose analysis includes a sampler and tubing for measurement at a sensor (not shown), and the pH and DO analysis includes optical sensing patches specific to the species and a reader. The DO, pH, glucose and DCO₂ measurements are conducted simultaneously as the sample passes through the flow cell.

FIG. 4A. An exploded view of a flow cell designed for gas measurements (e.g., DO and DCO₂).

FIG. 4B. An exploded view of a flow cell designed to measure pH and DO.

FIG. 4C. An exploded view of a flow cell designed to measure pH and diffusible soluble analytes (e.g., glucose).

FIG. 5A. An embodiment of a flow cell assembly for measurements of DO, DCO₂, pH and diffusible soluble analytes (e.g., glucose).

FIG. 5B. An embodiment of a flow cell assembly for measurements of DO, DCO₂, and pH.

FIG. 6A. pH measurements through noninvasive technique (N=4). Comparison between measurement through control method and noninvasive method. The corrected ratio on the y-axis is the raw value of the pH sensor reading. The boxes extend from the 25th to the 75th percentile of each group's distribution of values, horizontal lines in the boxes indicate the median values, and vertical extending lines indicate the most extreme values of each group.

FIG. 6B. Response times in control and noninvasive methods when the solutions were added to the vessel in descending order of pH values. The error bars indicate the standard deviation of the corresponding data sets.

FIG. 6C. Response times in control and noninvasive methods when the solutions were added to the vessel in ascending order of pH values. The error bars indicate the standard deviation of the corresponding data sets.

FIG. 7A. Noninvasive pH measurements before and after long-term exposure of the membrane to the cell culture medium (N=4). Comparison of measurements conducted via noninvasive method before and after the exposure of the cellulose membrane to the cell culture medium. The boxes in this figure extend from the 25th to the 75th percentile of each group's distribution of values, horizontal lines in the boxes indicate the median values, and vertical extending lines indicate the most extreme values of each group.

FIG. 7B. Calculated response times for the noninvasive method before and after exposure of the cellulose membrane to the cell culture medium. The error bars in this figure indicate the standard deviation of the corresponding data sets.

FIG. 8. pH profiles obtained from CHO-K1 culture process. Comparison of the pH profile obtained from the noninvasive method and control method throughout the CHO-K1 culture process.

FIG. 9. pH profiles obtained from E. coli culture process. The pH measurements were obtained through noninvasive (red) and control (blue) methods throughout the E. coli culture process. Dissolved oxygen was also monitored during the process (green). The results show the effectiveness of the noninvasive technique for pH measurement

FIG. 10A. Comparison of DO profiles obtained from flow cell and control method.

FIG. 10B. pH profiles obtained from flow cell and control method.

FIG. 10C. Dissolved DCO₂ profiles obtained from the flow cell and via control method from inside the bioreactor.

FIG. 11A. Flow cell measurements in E. coli culture process. During the E. coli culture process, medium including cells were recirculated between the shake flask and measurement of DO was conducted through flow cell and control method. The figure shows that the profiles obtained from the flow cell are comparable with control measurements.

FIG. 11B. Flow cell measurements in E. coli culture process. During the E. coli culture process, medium including cells were recirculated between the shake flask and measurement of pH was conducted through flow cell and control method. The figure shows that the profiles obtained from the flow cell are comparable with control measurements.

FIG. 11C. Flow cell measurements in E. coli culture process. During the E. coli culture process, medium including cells were recirculated between the shake flask and measurement of DCO₂ was conducted through flow cell and control method. The figure shows that the profiles obtained from the flow cell are comparable with control measurements.

FIG. 12A. Oxygen measurements to test the sensor response. The sensor was periodically flushed with N₂ gas which shows up as the dip in the O₂ values. Once the N₂ gas flushing stops, the 02 in the T-flask vessels diffuses in the sensor chamber and was recorded as peak in the plot.

FIG. 12B. CO₂ measurements to test the sensor response. The sensor was periodically flushed with N₂ gas which shows up as the dip in the CO₂ values. Once the N₂ gas flushing stops, the CO₂ in the T-flask vessels diffuses in the sensor chamber and was recorded as peak in the plot.

The features and advantages of the invention are more fully illustrated by the following non-limiting example, wherein all components are used in a particular form to demonstrate the usability and practice.

### DETAILED DESCRIPTION, AND PREFERRED EMBODIMENTS THEREOF

The presently disclosed subject matter now will be described more fully hereinafter with reference to the accompanying Figures, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Figures. Therefore, it is to be understood that the presently disclosed subject matter is not to be limited to the specific embodiments disclosed and that modifications and other embodiments are intended to be included within the scope of the appended claims.

Following long-standing patent law convention, the terms “a,” “an,” and “the” refer to “one or more” when used in this application, including the claims. Thus, for example, reference to “a subject” includes a plurality of subjects, unless the context clearly is to the contrary (e.g., a plurality of subjects), and so forth.

Throughout this specification and the claims, the terms “comprise,” “comprises,” and “comprising” are used in a non-exclusive sense, except where the context requires otherwise. Likewise, the term “include” and its grammatical variants are intended to be non-limiting, such that recitation of items in a list is not to the exclusion of other like items that can be substituted or added to the listed items.

For the purposes of this specification and appended claims, unless otherwise indicated, all numbers expressing amounts, sizes, dimensions, proportions, shapes, formulations, parameters, percentages, quantities, characteristics, and other numerical values used in the specification and claims, are to be understood as being modified in all instances by the term “about” even though the term “about” may not expressly appear with the value, amount or range. Accordingly, unless indicated to the contrary, the numerical parameters set forth in the following specification and attached claims are not and need not be exact, but may be approximate and/or larger or smaller as desired, reflecting tolerances, conversion factors, rounding off, measurement error and the like, and other factors known to those of skill in the art depending on the desired properties sought to be obtained by the presently disclosed subject matter. For example, the term “about,” when referring to a value can be meant to encompass variations of, in some embodiments, ±100% in some embodiments±50%, in some embodiments±20%, in some embodiments±10%, in some embodiments±5%, in some embodiments±1%, in some embodiments±0.5%, and in some embodiments±0.1% from the specified amount, as such variations are appropriate to perform the disclosed methods or employ the disclosed compositions.

Further, the term “about” when used in connection with one or more numbers or numerical ranges, should be understood to refer to all such numbers, including all numbers in a range and modifies that range by extending the boundaries above and below the numerical values set forth. The recitation of numerical ranges by endpoints includes all numbers, e.g., whole integers, including fractions thereof, subsumed within that range (for example, the recitation of 1 to 5 includes 1, 2, 3, 4, and 5, as well as fractions thereof, e.g., 1.5, 2.25, 3.75, 4.1, and the like) and any range within that range.

The term “culturing” as used herein refers to the controlled growth of cells ex vivo and/or in vitro. As used herein, “culturing” includes the growth of cells during cell expansion, or cell engineering (e.g., transduction with a construct for expressing, for example, a CAR (chimeric antigen receptor) or a TCR (T-cell receptor)). In some embodiments, the cultured cells are obtained from a subject, e.g., a human subject or a nonhuman animal. In some embodiments, the cell culturing is intended to expand the number of cultured cells, e.g., to increase proliferation of the cells, in an artificial but controlled environment. As used herein, “cell engineering” refers to the targeted modification of a cell. In some embodiments, the cell engineering comprises viral genetic engineering, non-viral genetic engineering, introduction of receptors to allow for tumor specific targeting (e.g., a TCR, TCRm, and/or a CAR), introduction of one or more endogenous genes that improve T cell function, introduction of one or more synthetic genes that improve T cell function, or any combination thereof. The cell culture process is well known in the art and includes isolation and growth of the cells. Culture cells can be subsequently manipulated of cultured cells, e.g., passaging, transfection, and transduction. Applications of cell culture are numerous including, but not limited to, the production of a myriad of biological products produced by recombinant DNA in cell cultures and the manufacture of viral vaccines.

As used herein, a “fluid” can be a liquid and/or a gas. In some embodiments, the fluid further comprises solid materials such as cells, cell fragments, and other detritus that are typically present in cell culturing processes.

System and Method for Noninvasive Monitoring of a Species within a Container

In a first aspect, a system and method for noninvasive monitoring of a species within a container is described. The system comprises the container, wherein at least a portion of said container comprises a membrane that permits the passage of said species therethrough. Membranes that permit the passage of specific species therethrough are well known in the art. For example, anion-exchange membranes and cation-exchange membranes in contact with a fluid (e.g., a liquid and/or a gas) permit the passage of anions and cations therethrough, respectively, without permitting the passage of the other components of a fluid. In some embodiments, the membranes described herein permit the passage of at least one of protons, oxygen, carbon dioxide, glucose, glutamine, enzymes, glutamine, phosphate, peptides, proteins (including antibodies), lactate, acetate, ammonia, amino acids, oxidizing agents, reducing agents, ions, micronutrients, cytokines, and other diffusible soluble analytes. In some embodiments, the membranes comprise at least one of polysulfone, silicone, cellulose, polyethersulfone, polyarylethersulfone, polyamide, polymethyl methacrylate, polyimide, polyester, polyvinylpyrrolidone, polycarbonate, polyacrylonitrile, polyethyleneimine, polytetrafluoroethylene (PTFE), sulfonated tetrafluoroethylene-based fluoropolymer-copolymer (e.g., Nafion™), or a blend thereof, which allows the transport of ions or small molecules. It is understood that the membrane is chosen based on the species it permits to pass therethrough and so other membranes are conceivable. In some embodiments, the cells within the container do not substantially adhere to the membrane. In some embodiments, the cells within the container do adhere to the membrane but the adherence has no substantial effect on the monitoring/measurement. In some embodiments, for measuring pH and analytes such as glucose, the membrane comprises cellulose with varying molecular weight cutoffs. In some embodiments, for measuring oxygen and carbon dioxide, the membrane comprises silicone or other gas permeable materials such as polytetrafluoroethylene (e.g., TEFLON™ AF 2400 or TEFLON™ AF 1600).

In some embodiments, the container is used for culturing cells, wherein knowing the concentration or amount of certain species in the fluid within the container is important to understanding the cell culture process. In some embodiments, the container is a cell culture flask, e.g., a T-flask. In some embodiments, the container is a bioreactor, e.g., a G-REX® bioreactor. In some embodiments, the container comprises polystyrene. In some embodiments, the container comprises a flat bottom shape. In some embodiments, the container comprises a plug seal closure.

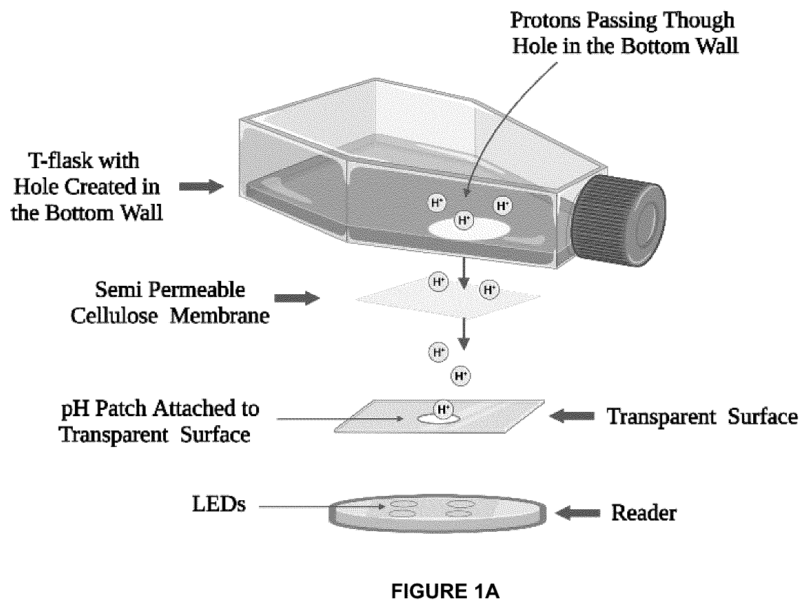

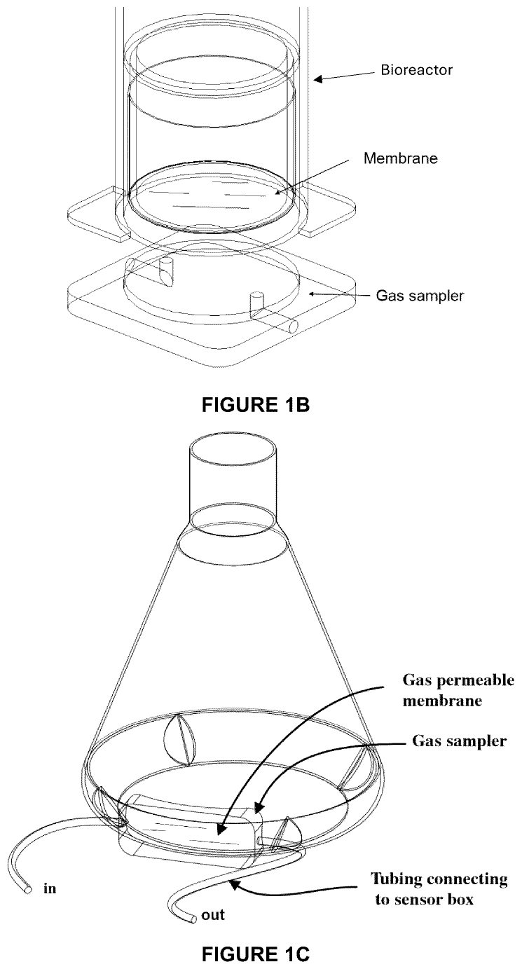

The system of the first aspect comprises the positioning of the membrane in/on the container. Referring to the illustration in FIG. 1A, in some embodiments, a hole or port can be created in the wall of a container, e.g., a cell culture flask. Thereafter, the membrane can be affixed to the container such that the hole or port is covered with said membrane, e.g., on the exterior of the container, and the hole or port is sealed. As discussed herein, the membrane will allow passage of a specific species therethrough, while ensuring that the remainder of the fluid remains in the container. In some embodiments, the membrane ensures a sterile environment within the container. The placement of the membrane over the hole is well understood by the person skilled in the art. In some embodiments, the membrane is “welded” to the container such that a fluid-tight seal is provided. Plastic “welding” is well understood in the art as a process of bringing two softened materials in contact to join or unite them, optionally with the use of additional heat. Upon cooling the different materials will be united. In some embodiments, the membrane is positioned within a ring, wherein the ring fits within the hole or port of the container such that a fluid-tight seal is provided. In some embodiments, the container, e.g., a bioreactor such as a G-REX® bioreactor, already comprises a membrane (see, e.g., FIG. 1B). It should be appreciated that the wall of the container can be any portion of the container that readily accommodates the placement of a membrane. For example, FIG. 1C illustrates an embodiment wherein a shake flask comprises a gas-permeable membrane and the species are monitored and measured using a gas sampler, as will be described below.

In some embodiments, the container can comprise a single membrane for the monitoring of a single species. In some other embodiments, the container can comprise a single membrane for the monitoring of multiple different species. For example, a large portion of the container can comprise a membrane, e.g., silicone, and both 02 and CO₂ can pass through said membrane and both DO and DCO₂ can be monitored simultaneously. In some other embodiments, the container can comprise more than one different membrane for the monitoring of multiple different species. For example, one membrane can comprise cellulose and pH is monitored and another membrane can comprise silicone and DO can be monitored.

The system further comprises at least one sensing device to monitor the species of interest and a detecting device for detecting the presence of the species of interest at the at least one sensing device.

In some embodiments, the at least one sensing device is an optical sensing patch, for example a pH sensing patch, a DO sensing patch or a DCO₂ sensing patch, as known in the art. Any type of sensing material known in the art can be used for the optical sensing patch as long as it emits light with at least one property (e.g., intensity, intensity decay rate) that is dependent on the amount of the species that is being measured and/or monitored. Advantageously, the use of an optical sensing patch allows for the measurement/monitoring to be conducted without requiring direct contact with the cell culture medium, i.e., is noninvasive. Moreover, the sampling approach is cost-effective and compatible with various types of single-use vessels. In some embodiments, for noninvasive monitoring of DO, an optical O₂ sensing patch is placed on, or in proximity to, the membrane. In some embodiments, when measuring DO, the membrane comprises silicone. During the process, oxygen diffuses through the membrane and is detected by the optical O₂ sensing patch. In some embodiments, for noninvasive monitoring of DCO₂, an optical CO₂ sensing patch is placed on, or in proximity to, the membrane. In some embodiments, when measuring DCO₂, the membrane comprises silicone. In some embodiments, the noninvasive technique for monitoring DCO₂ works based on measuring the initial diffusion rate of CO₂ through a silicone membrane in the wall of the container. In some embodiments, for noninvasive monitoring of pH, an optical pH sensing patch is placed on, or in proximity to, the membrane. In some embodiments, when measuring pH, the membrane comprises cellulose.

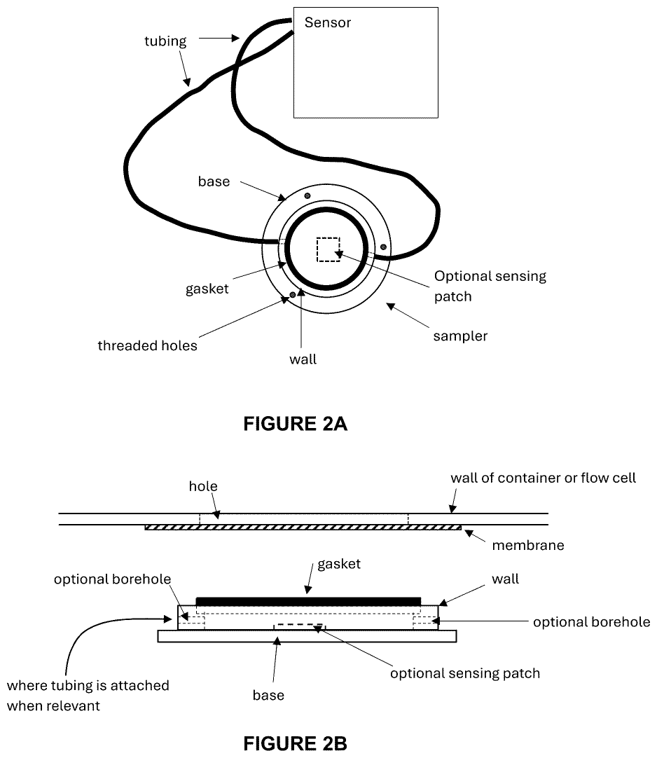

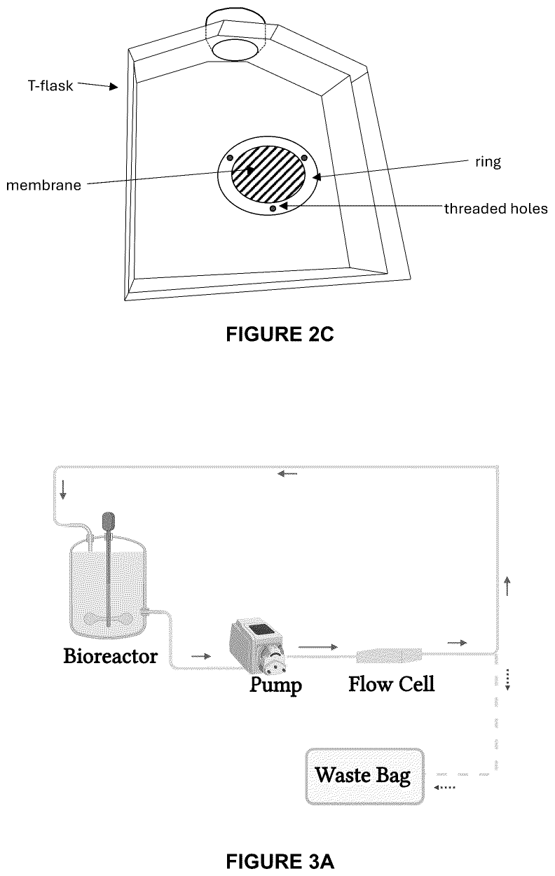

In some embodiments, the optical sensing patch is positioned directly on the membrane, e.g., by affixing or positioning the optical sensing patch on the membrane of the container. In some embodiments, the optical sensing patch is sandwiched between the membrane and an optically transparent layer (see, for example FIG. 1A), and hence is directly on the membrane. It should be appreciated that the optically transparent layer should not permit the passage of the species of interest therethrough. In some other embodiments, a sampler is utilized, wherein the sampler comprises a base, a gasket, and an optical sensing patch is positioned on the base within the gasket (see, e.g., FIGS. 2A and 2B for an embodiment of a sampler). As shown in FIGS. 2A and 2B, the sampler base can further comprise a sampler wall. The gasket can fit within the sampler wall such that the gasket has a height greater than the sampler wall to effectuate a tight fit with the membrane and wall of the container or flow cell (see, e.g., FIG. 2B). It should be appreciated that the sampler is made of a material that ensures that the species of interest does not pass through the material or otherwise is adsorbed on the material. In some embodiments, the diameter of the gasket of the sampler is greater than, or equal to, the diameter of the hole or port of the container. Upon placement of the sampler at the membrane, the sampler comprising a gasket insures that any species passing through the membrane, e.g., O₂ and/or CO₂ gas, are contained within the sampler. When an optical sensing patch is positioned on the base, i.e., “in proximity” of the membrane, the optical sensing patch can effectively monitor the presence of the species passing through the membrane and captured within the sampler. In some embodiments, “in proximity” corresponds to a distance between the membrane and the optical sensing patch of no more than about 0.1 mm to 2 mm, or about 0.1 mm to about 1 mm, or about 0.1 mm to about 0.5 mm. It should be appreciated that if an optical sensing patch is positioned in the sampler, the sampler should not include the boreholes (or they should be otherwise plugged). In some embodiments, the sampler is manufactured from an optically transparent material. It should be appreciated that although the sampler is shown in the figures as having a circular profile, it is not limited as such and can be square, rectangular, elliptical, polygonal, triangular, etc. In some embodiments, the volume of the sampler is in a range from about 0.5 mm³ to about 5 mm³, is configuration dependent, and is optimized based on the species to be measured.

When the at least one sensing device comprises an optical sensing patch, a detection device for monitoring the presence of the species of interest can comprise at least one radiation (e.g., light) source and at least one detector. In some embodiments, the radiation source can be an LED having approximately the relevant wavelength of excitation. In some embodiments, the radiation source can be at least two LEDs having two different wavelengths of excitation for ratiometric detection. In some embodiments, the at least one detector comprises a photodiode. In some embodiments, the at least one radiation source and the at least one detector are positioned in a reader for placement of the reader in proximity to the optical sensing patch (e.g., on the optically transparent layer or on the sampler). In some embodiments, the detection device further comprises optoelectronic components and controllers for the amount of species present.

In some embodiments, the at least one sensing device comprises the previously described sampler, along with tubing and a detection device (e.g., a sensor). Upon placement of the sampler (e.g., without the optical sensing patch but with the at least one borehole) at the membrane, the sampler ensures that any species passing through the membrane, e.g., O₂ and/or CO₂ gas, are contained within the sampler. The sampler can be in fluid communication with the detection device (e.g., the sensor) using tubing via the boreholes. For example, as shown in FIGS. 2A and 2B, the tubing can be attached at the sampler wall, which has a borehole therethrough so that any species trapped in the sampler can pass through the boreholes to the tubes and eventually to the sensor for detection. In some embodiments, the sensor further comprises a pump, e.g., a micropump, to bring gases (O₂ and/or CO₂) from the sampler to the sensor for measurement and optionally to pump gases (e.g., N₂, O₂, CO₂) to the sampler for passage through the membrane into the container to oxygenate or maintain the buffering capacity of the medium in the container. Sensors for determining the amount of oxygen or carbon dioxide in a gas sample are well known in the arts and are not intended to be limited herein. For example, O₂ can be measured using electrochemical or luminescent techniques and CO₂ can be measured using non-dispersive infrared (NDIR) techniques. In some embodiments, the detection device or sensor will exhibit at least one property (e.g., light emission intensity, light absorption, etc.) that is dependent on the amount of the species that is being measured and/or monitored and can be any sensor known in the art for detecting the species to be detected. In some embodiments, the sampler is manufactured from an optically transparent material. In some embodiments, when a measurement is ready to be made, a flush sequence can be initiated to flush the sampler with nitrogen. This flush sequence is preferably performed until optoelectronic components that are measuring signals from the detection device output a zero reading (after accounting for instrument offsets). This ensures constant initial conditions for all measurements. Once the flush sequence is complete, a measurement sequence is initiated wherein nitrogen is circulated through the sampler to the detection device/sensor. A measurement relating to the amount of species of interest present can then be obtained. The detection device can be based on any known means of detection including, but not limited to, optical, optoelectronic, electrochemical, acoustic, enzymatic, affinity, or optoelectronic detectors. In some embodiments, the detection device further comprises optoelectronic components and controllers for the amount of species present.

It should be appreciated that other sensing devices can be used and the optical sensing patches, the samplers and the measurement sensors described herein are not intended to limit the invention in any way.

In some embodiments of the first aspect, a ring is adapted to fit within or over the hole or port of the container. In some embodiments, the ring comprises a membrane positioned therein. In some embodiments, the ring comprises a connecting mechanism which can mate with an connecting mechanism on the sampler such that the sampler can be more easily and consistently attached to the ring of the container for monitoring of the species. For example, as shown in FIGS. 2A and 2C, the ring and the sampler comprise threaded holes such that a screw can be used to attach the sampler to the ring for a more precise and consistent fit over the membrane. It should be appreciated that the connecting mechanism embodiment illustrated in FIGS. 2A and 2C is not intended to limit the connecting mechanism and other connecting mechanisms are well known in the art and can be used instead. In some other embodiments, the container of the first aspect is not connected to the sampler at all.

In some embodiments, the system and method of the first aspect is qualitative. In some embodiments, the system and method of the first aspect is quantitative.

Accordingly, in some embodiments of the first aspect, a system for noninvasively detecting and quantitating species within a container is described, said system comprising:
the container, wherein the container comprises at least one hole or port in a wall of the container, wherein a membrane covers, or is positioned within, each hole or port, and wherein the membrane permits the passage of the species to be detected and quantitated therethrough;
at least one sensing device, wherein the at least one sensing device is positioned on, or in proximity to, the membrane, wherein the at least one sensing device is not in contact with any fluid present in the container, and wherein the at least one sensing device collects the species that pass through the membrane, reacts with the species that pass through the membrane, or both; and
a detection device for quantitating the species collected in, reacted with, or both collected in and reacted with, the at least one sensing device.

In some embodiments, a method of using the system of the first aspect is described, wherein the presence and/or an amount of a species in a fluid within a container is determined. The use of sensing devices and detection devices are well known in the art. Advantageously, the method is noninvasive.

In some embodiments, the detection device can be based on any known means of detection including, but not limited to, optical, optoelectronic, electrochemical, acoustic, enzymatic, affinity, or optoelectronic detectors. In some embodiments, the detection device further comprises optoelectronic components and controllers for the amount of species present. In some embodiments, the detection device data will also enable machine learning and artificial intelligence adoption in biomanufacturing.

Advantageously, the system and method for noninvasive monitoring of a species within a container addresses the major challenges associated with existing monitoring systems. One major advantage of this technology is the elimination of contamination risks. This is specifically important in the manufacturing process of cell therapies, where maintaining a contamination-free process in compliance with GMP regulations is critical. Furthermore, using the system and method of the first aspect, the risk of cytotoxicity associated with sensing parts is minimized because measurements are conducted through membranes, and no unwanted chemicals directly contact the cell culture medium. The technique can be utilized in cultures with different working volumes, accommodating a wide range of processes using various types of containers. Another advantage of the system and method of the first aspect is the ease of replacing the malfunctioning parts without interrupting the cell culture process. Accordingly, the sensing and detecting devices can easily be removed, replaced or recalibrated without compromising sterility within the container because faulty sensing devices have to be accessed. This cannot be done with conventional prior art systems because the sensing devices are inside of the containers.

### Flow Cells and Flow Cell Assemblies

In a second aspect, a flow cell system for simultaneously monitoring at least one species is described. The flow cell conducts online and simultaneous monitoring of process parameters from outside of the vessel, e.g., cell culture vessel, and the measurements are based on individual noninvasive methods developed for each analyte. In some embodiments, the species to be noninvasively monitored include, but are not limited to, at least one of pH, O₂, CO₂, glucose, glutamine, phosphate, peptides, proteins (including antibodies), lactate, acetate, ammonia, amino acids, oxidizing agents, reducing agents, ions, micronutrients, cytokines, and other diffusible soluble analytes. In some embodiments, the flow cell is microfluidic.

The flow cell technology addresses the major challenges associated with existing monitoring systems. One major advantage of this technology is the elimination of contamination risks. This is specifically important in the manufacturing process of cell therapies, where maintaining a contamination-free process in compliance with GMP regulations is critical. Furthermore, using the flow cell technology described herein, the risk of cytotoxicity associated with sensing parts is minimized because measurements are conducted through membranes, and no unwanted chemicals directly contact the cell culture medium. The technique can be utilized in cultures with different working volumes, accommodating a wide range of processes using various types of bioreactors. Another advantage of the flow cell system described herein is the ease of replacing the malfunctioning parts without interrupting the cell culture process. Accordingly, the sensing and detecting devices can easily be removed, replaced or recalibrated without compromising sterility or halting the process to dissemble to access faulty sensing devices. This cannot be done with conventional prior art systems because the sensing devices are inside of the containers and flow cells.

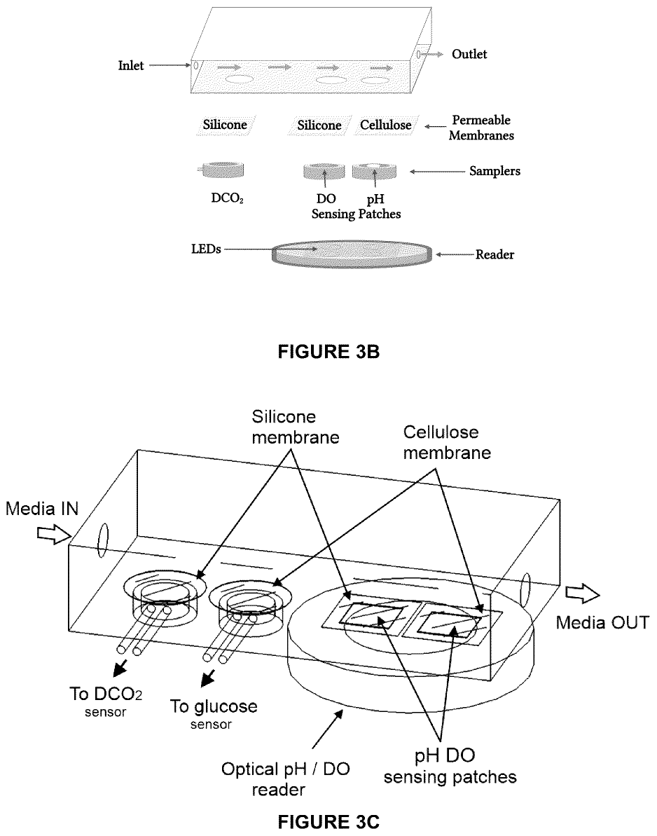

An embodiment of a flow cell system is illustrated in FIG. 3A. The flow cell is communicatively connected to a vessel, e.g., a bioreactor, comprising cells in a stage of the cell culture process. As illustrated in FIG. 3A, at least one pump is positioned between the vessel and the flow cell. Following passage through the flow cell, the fluid can be returned to the vessel and/or directed to waste. Vessels contemplated herein include, but are not limited to, T-flasks, petri dishes, cell factories, cell stack vessels, shake flasks, culture bags, roller bottles, stacked vessels (e.g., HYPERStack® Vessels), stir tanks, packed-bed bioreactor systems, and bioreactors. It should be appreciated however that if the vessel of the second aspect comprises a cell culture flask, e.g., a T-flask, it does not have a hole or port with a membrane positioned on or in the hole, e.g., as described in the first aspect. Although not shown, the flow cell system can further comprise additional pumps, valves, intake ports, exhaust ports, etc.

It should be appreciated that although shown in a recirculation loop in FIG. 3A, the flow cell can be positioned anywhere to measure species, e.g., downstream or upstream of any vessel, in the waste line (e.g., for sensitive cultures where contamination risk is high), etc. In other words, the positioning of the flow cell shown in the flow cell system of FIG. 3A is only for the purposes of providing an example and is not intended to limit where the flow cell can be positioned for measurements relating to the cell culturing process.

In some embodiments, the flow cell system can be operated in a batch, e.g., static, operation. In some embodiments, the flow cell system can be operated in a continuous, e.g., flow-through, operation.

Embodiment of a flow cell for the flow cell system are shown in FIGS. 3B and 3C. As shown in FIG. 3B, the flow cell has an inlet for entry of a medium to be monitored/measured, and an outlet for egress of said medium following monitoring/measuring. As shown in in FIG. 3B, the flow cell comprise three holes or ports, wherein analogous to the first aspect, the holes are covered by a membrane and any monitoring/measurement occurs noninvasively, i.e., external to the flow cell. The flow cell of FIG. 3B includes membranes positioned in/on the flow cell at the holes. Referring to FIG. 3B, the membrane can be affixed to the flow cell such that the hole is covered with said membrane, e.g., on the exterior of the flow cell, and the hole is sealed. As discussed herein, the membrane will allow passage of a specific species therethrough, while ensuring that the remainder of the fluid remains in the flow cell. The placement of the membrane over the hole is well understood by the person skilled in the art. In some embodiments, the membrane is “welded” to the flow cell such that a fluid-tight seal is provided. In some embodiments, the membrane is positioned within a ring, wherein the ring fits within the hole of the flow cell such that a fluid-tight seal is provided. In some embodiments, the flow cell of FIG. 3B can comprise one, two, three, four, five, or more dedicated monitoring/measurement holes or ports comprising a dedicated membrane covering. In some embodiments, the membranes covering more than one monitoring/measurement holes can be the same as or different from one another. In some embodiments, the flow cell can comprise a single hole with a single membrane for the monitoring of a single species. In some other embodiments, the flow cell can comprise a single hole with a single membrane for the monitoring of multiple different species. For example, a single hole covered by a silicone membrane can be used to monitor both DO and DCO₂ simultaneously. In some other embodiments, the flow cell can comprise more than one different membrane for the monitoring of multiple different species from more than one hole. For example, as shown in FIG. 3B, a flow cell with three different holes or ports can comprise one hole with a cellulose membrane for the monitoring of pH and two separate holes both with silicone membranes for the monitoring of DO and DCO₂. In another embodiments, as shown in FIG. 3C, a flow cell with four different holes or ports can comprise one hole with a cellulose membrane for the monitoring of pH, one hole with a cellulose membrane for monitoring of glucose, and two holes both with silicone membranes for the monitoring of DO and DCO₂. Although not illustrated in FIGS. 3B and 3C, the interior of the flow cell can comprise a substantially straight, non-tortuous path, from the inlet to the outlet or can comprise an opening or cavity positioned between the inlet and outlet, wherein the opening or cavity can be circular, elliptical, square, rectangular, trapezoidal, triangular or polygonal in shape.

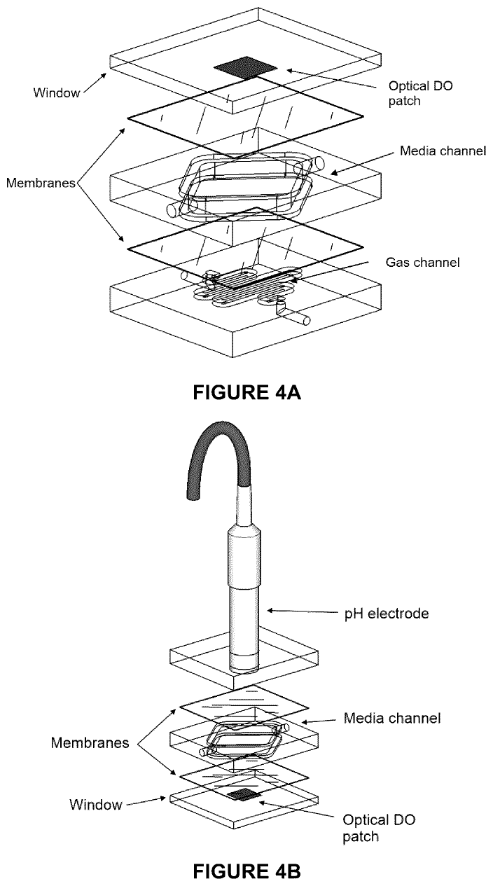

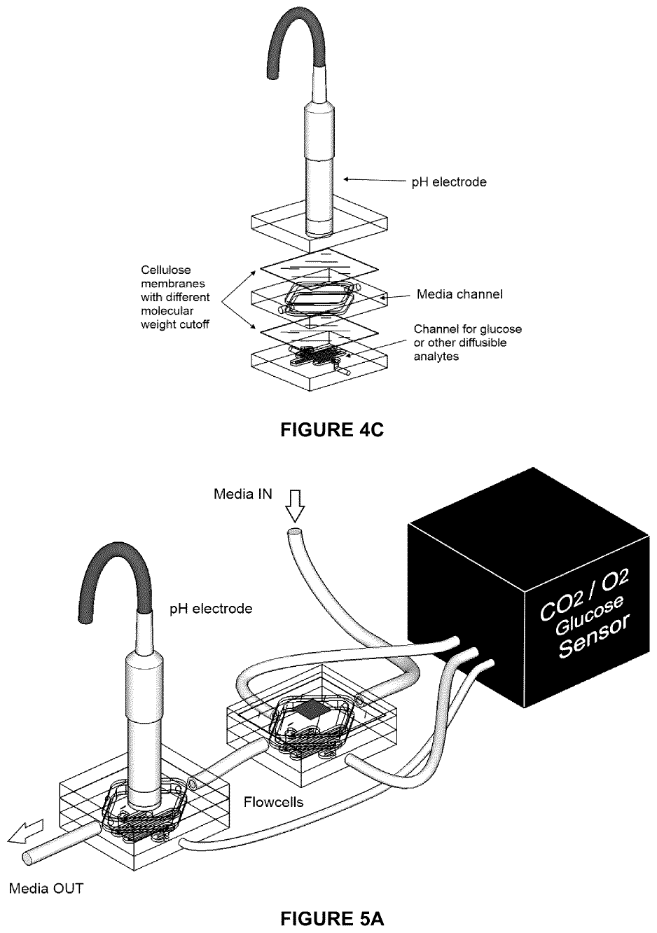

Another embodiment of a flow cell is illustrated in FIG. 4A, which is an exploded view of a flow cell designed for gas sensing (e.g., DO and DCO₂). In FIG. 4A, the flow cell comprises at least five layers: an optically transparent layer, a first membrane, a layer comprising a media channel, a second membrane, and a layer comprising a flow channel. In some embodiments, as shown in FIG. 4A, an optical sensing patch (e.g., an optical DO patch) is positioned between the optically transparent layer and the first membrane, and hence is directly on the membrane. It should be appreciated that the optical sensing patch could sense other species such as pH or DCO₂. In some embodiments, as shown in FIG. 4A, the media channel is a trapezoidal hole, wherein the shape is chosen to minimize the dead volume or no-flow zone formation in the media channel. As shown in FIG. 4A, the layer comprising the media channel further comprises ports that can be attached to tubing for communicative connection to the vessel such that fluid from the vessel can flow through the media channel. In some embodiments, as shown in FIG. 4A, the flow channel is serpentine in shape and is engraved into the layer such that the gas enters the flow channel through the second membrane and flows to the sensor using the micropump. It should be appreciated that the shape of the flow channel is not limited to a serpentine-shaped channel. In some embodiments, the flow channel is used to monitor/measure gases such as DO and/or DCO₂.

Another embodiment of a flow cell is illustrated in FIG. 4B, which is an exploded view of a flow cell designed for pH sensing and DO sensing. In FIG. 4B, the flow cell comprises at least five layers: a layer for positioning of an ion-selective electrode (e.g., a flat-tip pH electrode that is single or dual junction), a first membrane, a layer comprising a media channel, a second membrane, and an optically transparent layer. In some embodiments, the layer for positioning of an ion-selective electrode comprises a hole to bring the ion-selective electrode it in contact with the first membrane (e.g., a cellulose membrane). In some embodiments, the ion-selective electrode is selected from pH, phosphate, magnesium, ammonium, calcium, sodium, potassium, carbonate, sulfate, and fluoride. In some embodiments, as shown in FIG. 4B, the media channel is a trapezoidal hole, wherein the shape is chosen to minimize the dead volume or no-flow zone formation in the media channel. As shown in FIG. 4B, the layer comprising the media channel further comprises ports that can be attached to tubing for communicative connection to the vessel such that fluid from the vessel can flow through the media channel. In some embodiments, as shown in FIG. 4B, an optical sensing patch (e.g., an optical DO patch) is positioned between the second membrane and the optically transparent layer, and hence is directly on the membrane. It should be appreciated that the optical sensing patch could sense other species such as pH or DCO₂.

Another embodiment of a flow cell is illustrated in FIG. 4C, which is an exploded view of a flow cell designed for pH sensing and sensing of glucose or other diffusible analytes. In FIG. 4C, the flow cell comprises at least five layers: a layer for positioning of an ion-selective electrode, a first membrane, a layer comprising a media channel, a second membrane, and a layer comprising a flow channel. In some embodiments, the layer for positioning of an ion-selective electrode comprises a hole to bring the ion-selective electrode it in contact with the first membrane (e.g., a cellulose membrane). In some embodiments, the ion-selective electrode is selected from pH, phosphate, magnesium, ammonium, calcium, sodium, potassium, carbonate, sulfate, and fluoride. In some embodiments, as shown in FIG. 4C, the media channel is a trapezoidal hole, wherein the shape is chosen to minimize the dead volume or no-flow zone formation in the media channel. As shown in FIG. 4C, the layer comprising the media channel further comprises ports that can be attached to tubing for communicative connection to the vessel such that fluid from the vessel can flow through the media channel. In some embodiments, as shown in FIG. 4C, the flow channel is a serpentine-shaped channel engraved into the layer and is separated from the media channel by a cellulose membrane. When measuring soluble analytes such as glucose, glutamine, or phosphate, a known buffer flows through the flow channel and the diffusible analytes diffuse through the cellulose membrane into the buffer. In some embodiments, the buffer further comprises at least one additional compound to assist with the measurement at the detection device including, but not limited to, binding proteins, chelating agents, oxidants, reductants, metal ions, and any combination thereof. The buffer brings the diffused analytes to a detection device, i.e., the sensor, where the measurements can take place.

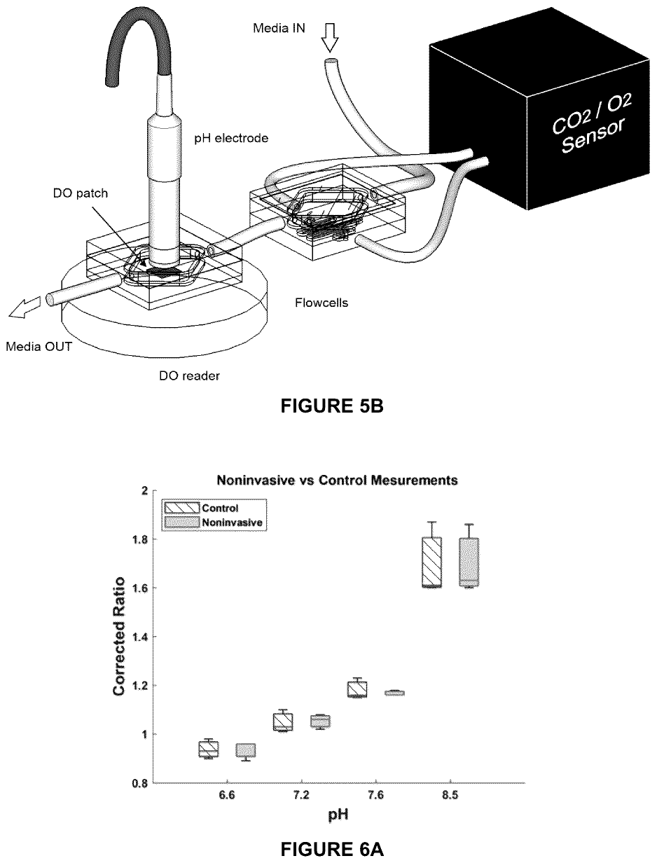

In some other embodiments, two or more flow cells are arranged in series, wherein fluid from the vessel flows into a first flow cell and then to a second flow cell, before being directed back to the vessel and/or waste. An embodiment of flow cells in series is shown in FIG. 5A, comprising a flow cell of FIG. 4A and a flow cell of FIG. 4C fluidly connected in series. Although not shown, a reader can be positioned in contact with the optically transparent layer of the flow cell of FIG. 4A (on the left) so that measurements at the optical sensing patch can be obtained. Another embodiment of flow cells in series is shown in FIG. 5B, comprising a flow cell of FIG. 4A and a flow cell of FIG. 4B fluidly connected in series. As shown in FIG. 5B, a reader can be positioned in contact with the optically transparent layer of the flow cell of FIG. 4B (on the right) so that measurements at the optical sensing patch can be obtained. It should be appreciated that other combinations of flow cells in series is contemplated and that FIGS. 5A and 5B are just examples of the versatility of the flow cells and how they can be arranged.

For the flow cells, the membrane permits the passage of species to be monitored/measured therethrough. Membranes that permit the passage of specific species therethrough are well known in the art. It is therefore appreciated that the membranes can be the same as or different from one another. In some embodiments, the membranes described herein permit the passage of at least one of protons, oxygen, carbon dioxide, glucose, glutamine, phosphate, peptides, proteins (including antibodies), lactate, acetate, ammonia, amino acids, oxidizing agents, reducing agents, ions, micronutrients, cytokines, and other diffusible soluble analytes. In some embodiments, the membranes comprise at least one of polysulfone, silicone, cellulose, polyethersulfone, polyarylethersulfone, polyamide, polymethyl methacrylate, polyimide, polyester, polyvinylpyrrolidone, polycarbonate, polyacrylonitrile, polyethyleneimine, polytetrafluoroethylene (PTFE), sulfonated tetrafluoroethylene-based fluoropolymer-copolymer (e.g., Nafion™), or a blend thereof, which allows the transport of ions or small molecules. It is understood that the membrane is chosen based on the species it permits to pass therethrough and so other membranes are conceivable. In some embodiments, any cells passing through the flow cell do not substantially adhere to the membrane(s). In some embodiments, any cells passing through the flow cell do adhere to the membrane(s) but the adherence has no substantial effect on the monitoring/measurement.

The flow cells further comprise at least one sensing device to monitor the species of interest and a detection device of detecting the presence of the species of interest at the at least one sensing device. In some embodiments, the detection device can be based on any known means of detection including, but not limited to, optical, optoelectronic, electrochemical, acoustic, enzymatic, affinity, or optoelectronic detectors. In some embodiments, the detection device further comprises optoelectronic components and controllers for the amount of species present.

In some embodiments, the at least one sensing device is an optical sensing patch, for example a pH sensing patch, a DO sensing patch or a DCO₂ sensing patch, as described herein in the first aspect. Advantageously, the use of an optical sensing patch allows for the measurement/monitoring to be conducted without requiring direct contact with the cell culture medium, i.e., is noninvasive. In some embodiments, optical sensing patchs are placed on, or in proximity to, the membrane. In some embodiments, the optical sensing patch is positioned directly on the membrane, e.g., by affixing or positioning the optical sensing patch on the membrane of the flow cell. In some embodiments, the optical sensing patch is sandwiched between the membrane and an optically transparent layer (see, for example FIG. 1A), and hence is directly on the membrane. It should be appreciated that the optically transparent layer should not permit the passage of the species of interest therethrough. In some other embodiments, a sampler, as described hereinabove in the first aspect, is utilized. Upon placement of the sampler at the membrane, the sampler comprising a gasket insures that any species passing through the membrane, e.g., O₂ or CO₂ gas, are contained within the sampler. If an optical sensing patch is positioned on the base of the sampler, i.e., “in proximity” of the membrane, the optical sensing patch can effectively monitor the presence of the species passing through the membrane and captured within the sampler. In some embodiments, “in proximity” corresponds to a distance between the membrane and the optical sensing patch of no more than about 0.1 mm to 2 mm, or about 0.1 mm to about 1 mm, or about 0.1 mm to about 0.5 mm.

When the at least one sensing device comprises an optical sensing patch, a detection device for monitoring the presence of the species of interest comprises at least one radiation (e.g., light) source and at least one detector. In some embodiments, the radiation source can be an LED having approximately the relevant wavelength of excitation. In some embodiments, the radiation source can be at least two LEDs having two different wavelengths of excitation for ratiometric detection. In some embodiments, the at least one detector comprises a photodiode. In some embodiments, the at least one radiation source and the at least one detector are positioned in a reader for placement of the reader in proximity to the optical sensing patch (see, e.g., FIGS. 3B and 5B), for example in contact with the optically transparent layer. In some embodiments, the detection device further comprises optoelectronic components and controllers for the amount of species present.

In some embodiments, the at least one sensing device comprises the previously described sampler, along with tubing and a detection device (e.g., a sensor). Upon placement of the sampler at the membrane, the sampler ensures that any species passing through the membrane, e.g., O₂ or CO₂ gas, are contained within the sampler. The sampler can be in fluid communication with the detection device (e.g., the sensor) using tubing via the boreholes. For example, as shown in FIGS. 2A, 2B, and 3B, the tubing can be attached at the sampler wall, which has a borehole therethrough so that any species trapped in the sampler can pass through the boreholes to the tubes and eventually to the sensor for detection. In some embodiments, the sensor further comprises a pump, e.g., a micropump, to bring gases (O₂ and CO₂) from the sampler to the sensor. Sensors for determining the amount of oxygen or carbon dioxide in a gas sample are well known in the arts and are not intended to be limited herein. For example, O₂ can be measured using electrochemical or luminescent techniques and CO₂ can be measured using non-dispersive infrared (NDIR) techniques. In some embodiments, the detection device or sensor will exhibit at least one property (e.g., light emission intensity, light absorption, etc.) that is dependent on the amount of the species that is being measured and/or monitored and can be any sensor known in the art for detecting the species to be detected. In some embodiments, the sampler is manufactured from an optically transparent material. In some embodiments, when a measurement is ready to be made, a flush sequence can be initiated to flush the sampler with nitrogen. This flush sequence is preferably performed until optoelectronic components that are measuring signals from the detection device output a zero reading (after accounting for instrument offsets). This ensures constant initial conditions for all measurements. Once the flush sequence is complete, a measurement sequence is initiated wherein nitrogen is circulated through the sampler to the detection device/sensor. A measurement relating to the amount of species of interest present can then be obtained. In some embodiments, the detection device can be based on any known means of detection including, but not limited to, optical, optoelectronic, electrochemical, acoustic, enzymatic, affinity, or optoelectronic detectors. In some embodiments, the detection device further comprises optoelectronic components and controllers for the amount of species present.

In some embodiments, the at least one sensing device comprises an ion-selective electrode and the detection device is any meter known to communicate with the selected sensing device, e.g., a pH meter. In some embodiments, the ion-selective electrode is selected from pH, phosphate, magnesium, ammonium, calcium, sodium, potassium, carbonate, sulfate, and fluoride.

In some embodiments, the at least one sensing device comprises a flow channel, wherein the flow channel is engraved into a layer and has a shape that permits contact of fluid in the flow channel with the membrane (see, e.g., FIGS. 4A and 4C). In some embodiments, the flow channel is serpentine-shaped. In practice, the flow channel is positioned in contact with a membrane, so that any species present in a fluid passing through the media channel that are permitted to pass through the second membrane, e.g., O₂ or CO₂ gas or glucose, enter the flow channel. The flow channel is in fluid communication with the detection device (e.g., the sensor) using tubing. In some embodiments, the sensor further comprises a pump, e.g., a micropump, to bring gases (O₂ and CO₂) or liquid from the flow channel to the sensor. Sensors for determining the amount of oxygen or carbon dioxide in a gas sample or glucose and other diffusible soluble analytes are well known in the arts and are not intended to be limited herein. For example, O₂ can be measured using electrochemical or luminescent techniques and CO₂ can be measured using non-dispersive infrared (NDIR) techniques. In some embodiments, the detection device or sensor will exhibit at least one property (e.g., light emission intensity, light absorption, etc.) that is dependent on the amount of the species that is being measured and/or monitored and can be any sensor known in the art for detecting the species to be detected. In some embodiments, when a measurement is ready to be made, a flush sequence can be initiated to flush the flow channel with nitrogen. This flush sequence is preferably performed until optoelectronic components that are measuring signals from the detection device output a zero reading (after accounting for instrument offsets). This ensures constant initial conditions for all measurements. Once the flush sequence is complete, a measurement sequence is initiated wherein nitrogen is circulated through the flow channel to the detection device/sensor. A measurement relating to the amount of species of interest present can then be obtained. In some embodiments, the detection device can be based on any known means of detection including, but not limited to, optical, optoelectronic, electrochemical, acoustic, enzymatic, affinity, or optoelectronic detectors. In some embodiments, the detection device further comprises optoelectronic components and controllers for the amount of species present.

It should be appreciated that other sensing devices and other detection devices are contemplated and the examples provided herein are not intended to limit the flow cell or flow cell system in any way.

In some embodiments, the flow cell is made of material that cells do not substantially adhere to. In some embodiments, the flow cell comprises polystyrene.

Accordingly, in some embodiments of the second aspect, a flow cell for noninvasively detecting and quantitating species is described, said flow cell comprising:
a layer for positioning of a first sensing device;
a first membrane layer, wherein the first membrane layer permits the passage of a species to be detected by the first sensing device;
a layer comprising a media channel;
a second membrane layer, wherein the second membrane layer permits the passage of a species to be detected by a second sensing device; and
a layer for positioning of the second sensing device,
   wherein the layer comprising the media channel can be communicatively connected to a vessel comprising a medium, wherein the medium comprises species to be detected and quantitated.

In another embodiment of the second aspect, a system for noninvasively detecting and quantitating species in a medium contained in a vessel is described, said system comprising:
a flow cell;
a pump; and
a vessel,
   wherein the flow cell comprises:
a layer for positioning of a first sensing device;
a first membrane layer, wherein the first membrane layer permits the passage of a species to be detected by the first sensing device;
a layer comprising a media channel;
a second membrane layer, wherein the second membrane layer permits the passage of a species to be detected by a second sensing device; and
a layer for positioning of the second sensing device,
   wherein the layer comprising the media channel can be communicatively connected to the vessel comprising the medium for detection and quantitation of a species contained therein.

In another embodiment of the second aspect, a system for noninvasively detecting and quantitating species in a medium contained in a vessel is described, said system comprising:
a flow cell;
a pump; and
a vessel,
   wherein the flow cell is communicatively connected to the vessel comprising the medium for detection and quantitation of a species contained therein, and wherein the flow cell comprises:
a flow cell container, wherein the flow cell container comprises at least one hole or port in a wall of the flow cell container, an inlet and an outlet, wherein a membrane covers, or is positioned within, each hole or port, and wherein the membrane permits the passage of the species to be detected and quantitated therethrough;
at least one sensing device, wherein the at least one sensing device is positioned on, or in proximity to, the membrane, wherein the at least one sensing device collects the species that pass through the membrane, reacts with the species that pass through the membrane, or both; and
a detection device for quantitating the species collected in, reacted with, or both collected in and reacted with, the at least one sensing device.

In some embodiments, a method of using the flow cell or the system of the second aspect is described, wherein the presence and/or an amount of a species in a fluid within a vessel is determined by passing the fluid from the vessel through the flow cell. The use of sensing devices and detection devices are well known in the art. Advantageously, the method is noninvasive.

In some embodiments, the detection device data will also enable machine learning and artificial intelligence adoption in biomanufacturing.

The features and advantages of the invention are more fully illustrated by the following non-limiting examples, wherein all parts and percentages are by weight, unless otherwise expressly stated.

### EXAMPLE 1

### 1. Analytics

### A. Optical Measurement System

Optical sensors, comprising electronics and sensing patches, were utilized for measuring DO and pH. A pH sensing patch includes a fluorescent dye, 6,8-dihydroxypyrene-1,3-disulfonic acid disodium salt (DHDS), immobilized in a hydrogel matrix (Ge et al. 2012), wherein the excitation spectrum of the dye changes in response to variations in the pH of the solution (Vallejos et al. 2010). The technique for online measurement of pH is a ratio-metric method wherein the pH value of the media is correlated with the corrected ratio of the emission intensities at two excited wavelengths of 468 nm and 408 nm. In a study on low-cost calibration-free pH sensing (Ge et al. 2012), it was found that the brightness of the violet and blue LEDs used to build the detectors for excitation was not completely uniform, which could introduce inter-device differences. To solve this problem, an algorithm was introduced to correct the effect of LED brightness on the ratio of fluorescence intensities, referred to as the corrected ratio (Id.). Advantageously, this pH sensing patch is disposable and substantially calibration-free, which is possible because each individual pH sensing patch has the same composition as the large sheet it came from. This permits calibration of a few randomly selected sensor patches from a whole batch during manufacture. All others from the same batch can be used directly without need for individual calibration (Id.).

For the DO patch, the sensing properties of the fluorophore, tris-(bathophenanthroline) ruthenium (II) chloride, are known to be influenced by alterations in the DO concentration (Ge & Rao 2012; Tolosa et al. 2002).

For evaluation of the noninvasive method described herein, control and noninvasive measurements were conducted simultaneously. The DO and pH patches were autoclaved at 121° C. for 20 minutes before conducting measurements. For control measurements, the patches were attached inside the cell culture vessel (Ge & Rao 2012). For noninvasive measurements, the patches were attached to the samplers outside the cell culture vessel. The preparation of the samplers is described in later sections. To conduct measurements through control and noninvasive methods, readers were placed below the vessel, and the LED light was aligned with the sensing patches. During the process, in noninvasive techniques, oxygen and protons pass through the permeable membranes of silicone and cellulose, respectively. The LED light emitted by the readers is an excitation source. Upon excitation by the LED light, the dyes within the patch emit light which is detected, analyzed, and converted to the appropriate readings.

### B. Rate-Based Measurement System

A rate-based technique was utilized for conducting online measurement of CO₂ in the flow cell. This technique is based on correlating the CO₂ concentration in the cell culture medium with the diffusion rate of the CO₂ through the silicone membrane (Chatterjee et al. 2015). In this method, CO₂ passes through the silicone membrane, is collected in the sampler, and is transferred to the sensor for measurements. The method was previously evaluated band the results indicate the effectiveness of the technique (Rahmatnejad et al. 2022; Chatterjee et al. 2015).

### C. Sensor Calibration

The pH sensor calibration was conducted by attaching a pH sensing patch to the bottom wall of the vessel and introducing buffers with pH values ranging from 5.5 to 8.5. The corrected ratio corresponding to each pH value was measured and recorded. The relationship between pH value and corrected ratio was determined through regression interpolation.

The CO₂ sensor calibration was conducted by sparging different percentages of CO₂ (0.0%, 2.5%, 5.0%, 7.5%, 10.0% for mammalian cultures and 0.0%, 5%, 10.0%, 15.0%, 20.0% for microbial fermentation) into the medium. For DO sensor calibration, a DO sensing patch was attached to the bottom wall of the vessel. Subsequently, different percentages of O₂ were sparged into the medium by combining different percentages of air (0.0%, 20%, 40%, 60%, 80%, and 100%) and nitrogen. In both calibration processes, the gas mixtures were created using two mass flow controllers (Digital Pressure Controller, Single-Valve, 0-30 psia, Cole-Parmer, Vernon Hills, IL, USA).

The percentage of gases sparged into the medium was converted to the concentration of dissolved gases utilizing Henry's Law relation. Henry's law constants were obtained from the compilation of Henry's law constants (Sander, 2015). For each percentage of CO₂ sparged, the initial diffusion rate of CO₂ through the silicone membrane was measured using the LabVIEW software developed by the present inventors. Regression interpolation was then utilized to convert measurements into concentrations of the respective gases.

### 2. Noninvasive Monitoring of pH

### A. T-flask Setup Preparation

To prepare the setup (i.e., a modified T-flask) for noninvasive measurement of pH, a hole was created in the bottom wall of a T-flask. Subsequently, a semi-permeable cellulose membrane (Fisher Scientific, Hampton, NH, USA) was attached externally over the hole. A sampler, comprising a pH sensing patch attached to an optically transparent layer, was attached to the cellulose membrane externally (i.e., the pH sensing patch is not sandwiched between the optically transparent layer and the cellulose membrane). The sensing patch was aligned with the center of the hole. The semi-permeable cellulose membrane has a pore size of 4.8 nm and molecular weight cut off (MWCO) of 12000 Daltons allowing small-molecule components of the cell culture medium to move towards an equilibrium concentration on both sides of the membrane. Different parts of the modified T-flask are shown in FIG. 1A. To conduct the online measurement of pH, a reader was placed below the vessel so that the LED light is aligned with the pH sensing patch in the sampler. After preparing the noninvasive measurement setup, a pH sensing patch was attached inside to the bottom wall of a modified T-flask, as a control method for pH measurement. Different buffers with varying pH values were added to the T-flask. After adding each solution, the measurements through control and noninvasive methods were recorded. The response times, representing the time taken for the sensor to reach 90% of the output, were calculated for both methods.

### B. Long-Term Exposure of the Cellulose Membrane to the Medium

To investigate the impact of the exposure of the cellulose membrane to the cell culture medium on the pH measurements, pre-calibration (calibration before exposure to the cell culture medium) and post-calibration (calibration after exposure to the cell culture medium) were conducted through a noninvasive technique. After pre-calibration, 10 ml of complete medium comprising 10% v/v Fetal Bovine Serum (FBS) (ATCC, Manassas, VA, USA) and 90% v/v of Dulbecco's Modified Eagle's Medium (DMEM) (ATCC, Manassas, VA, USA) was added to a modified T-25 flask. The T-flask was then placed in the 5% CO₂ incubator and maintained for 10 days. On day 10, the medium was removed, and the T-flask was rinsed with deionized (DI) water before the post-calibration process was performed. The calibration processes were conducted based on the procedures described herein.

### C. Cell Attachment on Cellulose Membrane

The noninvasive pH measurements were conducted through a cellulose membrane. To study whether the pH measurements were affected by cell attachments in the culture process of adherent cells, cell attachment on the cellulose membrane was investigated by conducting DAPI (4′-6-di-amidino-2-phenylindole) staining on the membrane. For this purpose, 2 cm ×2 cm pieces of the cellulose membrane were placed in 6 wells of a 6-well-plate. In each well, adherent Chinese hamster ovary (CHO-K1) cells (ATCC, Manassas, VA, USA) were cultured in 3 ml of a complete medium composed of 10% v/v Fetal Bovine Serum (FBS) (ATCC, Manassas, VA, USA), and 90% v/v HAM's F12 medium with L-Glutamine (Lonza, Walkersville, MD, USA). The seeding density was 3.1×10⁴ cells/cm², and one piece of membrane was harvested each day on days 3, 4, 5, 6, 7 and 8. Cells on membranes were fixed in 4% Paraformaldehyde (TissuePro Technology, Gainesville, FL, USA), and membranes were stored in Phosphate Buffered Saline (PBS) (Thermo Fisher Scientific, Waltham, MA, USA). For DAPI staining, a 300 nM DAPI solution was prepared by dissolving the content of the vial in 2 ml of DI water and subsequent dilution in PBS. The cellulose membranes were stained by adding 300 μl of the diluted DAPI solution, followed by 5 minutes incubation and rinsing with PBS three times. The stained membranes were then imaged using a fluorescence microscope.

### D. Noninvasive pH Measurement in CHO-K1 Cell Culture Process

A modified T-flask, prepared based on the process explained in FIG. 1A, was used as a cell culture vessel in this experiment. The T-flask was designed to measure pH in a noninvasive way. As a control method for pH measurement, a pH sensing patch was attached inside the modified T-flask. Subsequently, CHO-K1 cells were seeded in the T-flask with a working volume of 53 ml and seeding density of 2.85×10⁴ cells/cm². The cell culture process was conducted in a 5% CO₂ incubator set at 37° C., and pH was simultaneously monitored through both control and noninvasive methods.

E. Noninvasive pH Measurement in E. coli Culture Process

Fifty μl of BL21 (DE3) E. coli (Invitrogen, Waltham, MA, USA) was added to 50 ml of LB Lennox medium in a 200 ml shake flask. The medium contained 10 g tryptone, 5 g yeast extract, and 5 g sodium chloride per liter. The cells were grown at 37° C. and 180 rpm for 20-24 hours. The setup described in FIG. 1A served as the cell culture vessel. For monitoring DO and pH through the control method, sensing patches were attached inside to the bottom wall of the T-flask. The pH measurements were simultaneously conducted through the noninvasive technique using the noninvasive measurement setup, e.g., as shown in FIG. 1A. In this experiment, the preculture was inoculated in the vessel, and an initial optical density (OD) of 0.65 in a working volume of 250 ml was achieved. The culture was conducted at an agitation speed of 200 rpm at 37° C., and 25 μl of Kanamycin was added to the T-flask to isolate the Escherichia coli (E. coli) bacteria.

### 3. Flow Cell

### A. Flow Cell Setup Preparation

To conduct the simultaneous monitoring of DO, pH, and DCO₂ from outside of the cell culture vessel, a flow cell technology was developed. The online measurements for different analytes were conducted as the sample passes through the flow cell. After the sample exits the flow cell, it is returned to the bioreactor or transferred to the waste bag. FIG. 3A shows the flow cell setup. The flow cell features three holes in the bottom wall (see, FIG. 3B). Two silicone membranes permeable to 02 and CO₂, and a cellulose membrane permeable to protons were attached to the holes externally. The flow cell and samplers were fabricated from acrylic sheets. The DCO₂ sampler includes a cavity in the center for collecting the CO₂ gas diffusing from the silicone membrane, and two channels, for transferring the gas to the sensor. DO and pH samplers are optically transparent layers with corresponding patches attached to them. All three samplers were externally attached to the membranes. To conduct online measurements, the flow cell was placed on the reader, and the DO and pH patches were aligned with the LED lights.

### B. Flow Cell Measurements

The LB broth medium was prepared by suspending 20 g of LB broth powder (Thermo Fisher Scientific, Waltham, MA, USA) in 1 L purified water. Different percentages of 02 and CO₂ were sparged in the LB broth medium. The medium was continuously recirculated between the T-175 flask and the flow cell utilizing a peristaltic pump. DO, pH, and DCO₂ were simultaneously measured from inside the flask and through the flow cell. The dimensions of the flow cell utilized were 9 cm L ×3 cm W ×1 cm H, and the flow rate for the sample was 0.25 ml/s.

For evaluating the pH measurements through the flow cell, a pH sensing patch was attached inside the T-flask as a control method. 200 ml of LB broth medium was added to the T-flask, and different percentages of CO₂ (0%, 10%, 20%, and 2.5%) were sparged into the medium. Online measurements through the flow cell and control method were simultaneously conducted while the medium was continuously recirculated between the flow cell and the cell culture vessel.

To evaluate the efficacy of the flow cell in measuring CO₂, 700 ml of LB broth medium was added to a vertically positioned T-175 flask. Various percentages of CO₂ (0%, 20%, 40%, 60%, 80%, and 100%) were sparged into the medium. The medium was continuously recirculated between the flow cell and the T-flask. Control measurements were obtained directly inside the vessel through the rate-based technique via a silicone sampling loop submerged in the cell culture medium, and simultaneous measurements were conducted through the flow cell.

For evaluating DO measurements through the flow cell, 200 ml of LB broth medium was added to the T-flask, and different percentages of 02 (20%, 15%, 10%, 5%, and 0%) were sparged into the medium. Flow cell and control measurements were simultaneously conducted while the medium was continuously recirculated between the flow cell and the cell culture vessel.

In all experiments, gas mixtures were created through two mass flow controllers (Digital Pressure Controller, Single-Valve, 0-30 psia, Cole-Parmer, Vernon Hills, IL, USA).

C. Flow Cell Measurements in E. coli Culture Process

DO and pH patches were attached to the inside of the bottom wall of the 2000 ml shake flask to conduct control measurements inside the culture. Subsequently, E. coli was inoculated into the shake flask with a working volume of 1000 ml. The agitation speed and temperature were set at 180 rpm and 37° C., respectively. The initial optical density (OD), measured at 600 nm, was 0.9. To measure DCO₂ through the control method, a silicone sampling loop was submerged in the cell culture medium, and the online measurements were conducted through the rate-based technique. The sample was continuously recirculated between the flow cell and the shake flask with a flow rate of 0.25 ml/s.

### D. Flow Cell Delay

The flow cell measurements are conducted by transferring the sample from the cell culture vessel to the flow cell outside the cell culture vessel. Therefore, a delay for flow cell measurements is expected. Different factors, such as the length of the transfer tube, flow rate, and volume of the flow cell, contribute to the delay in flow cell measurements. The time required for transferring the sample to the flow cell can be calculated using equation (1):

$$Q = \frac{A d}{t} \to t = \frac{A d}{Q} \qquad ( 1 )$$

where: Q is flow rate; A is area of the cross-section of the tube; d is length of the tube; and t is time. The residence time of sample in the flow cell could be calculated using equation (2):

$${R}_{T} = \frac{V}{Q} \qquad ( 2 )$$

where: R_{T} is residence time; V is volume of the flow cell; and Q is flow rate. According to Fick's second Law, equation (3) can be obtained which roughly estimates the time required for diffusion through membranes (Calculator Academy 2024):

$$Δ t \approx \frac{Δ {x}^{2}}{2 D} \qquad ( 3 )$$

where: Δt is time for diffusion of the specific analyte is see; Ax is thickness of the membrane; and D is diffusion coefficient.

### 4. Results and Discussion

### A. Noninvasive Measurement of pH

The technique for noninvasive monitoring of pH was developed by placing a cellulose membrane between the cell culture medium and a pH-sensing patch to yield a modified T-flask. During the process, protons diffuse through the membrane and contact the sensing patch. The measurements are subsequently conducted based on the method described herein. The efficiency of the technique was studied by adding solutions with different pH values to the modified T-flask.

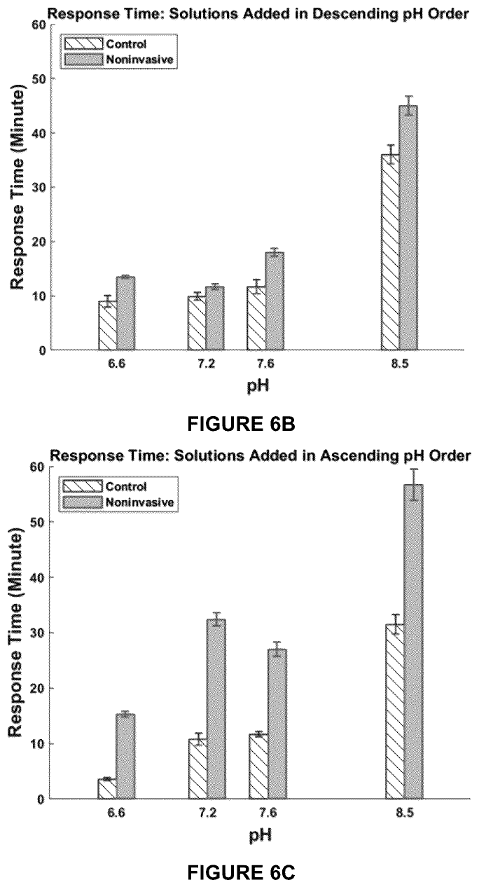

FIG. 6A demonstrates the measurements through the control and noninvasive methods. FIGS. 6B and 6C illustrate the response times for control and noninvasive methods when the solutions were added in descending order of pH values and ascending order of pH values, respectively. As it is shown in FIG. 6A, the measurements through noninvasive techniques are comparable with control measurements. The paired samples t-test was performed, and the calculated p-value of 0.9954 confirms that there is no significant difference between the control and noninvasive methods. Similarly, the p-values were obtained for response times in FIGS. 6B and 6C by performing paired samples t-test and the results are respectively 0.4408 and 0.0014. These results indicate that there is a significant difference between response times when solutions were added in ascending order which is due to the faster diffusion of protons into the patch compared to their outward diffusion. However, the results show no significant difference between the two methods when the solutions were added in descending order.

### B. Long-Term Exposure of the Cellulose Membrane to the Medium

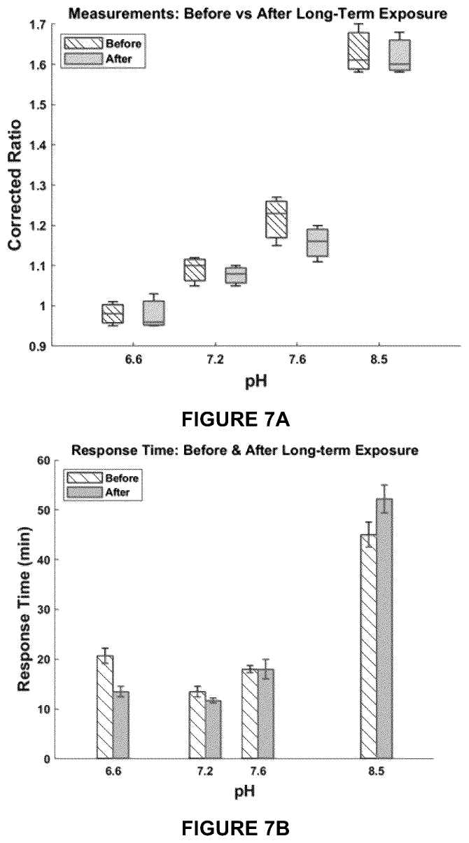

In the noninvasive method for monitoring pH, the cellulose membrane is in direct contact with the cell culture medium. A study was conducted to investigate the effect of long-term exposure of cellulose membrane to the cell culture medium on the pH measurements and the response time. FIGS. 7A and 7B, respectively, show the measurements and response times before and after exposure to the cell culture medium. The p-values obtained by performing paired t-test for data shown in FIG. 7A and FIG. 7B are 0.8583 and 0.8589, respectively. This indicates that there is no significant difference between the noninvasive measurements before and after the long-term exposure to the membrane. Therefore, the exposure of the membrane to the medium does not affect the measurements through the noninvasive method.

### C. Cell Attachment on Cellulose Membrane

In the noninvasive technique for measuring pH through cellulose membrane, the attachment of cells on the membrane was studied to investigate whether the measurements are affected by cell attachment when culturing adherent cells. Results from DAPI staining indicate no cell attachment on cellulose membranes on days 3, 4, and 5 of the culture process. On days 6, 7, and 8, only negligible cell attachment was found (not shown). These results indicate the effectiveness of the noninvasive technique for pH measurement in the culture process of adherent cells.

### D. Noninvasive pH Measurement in CHO-K1 Cell Culture Process

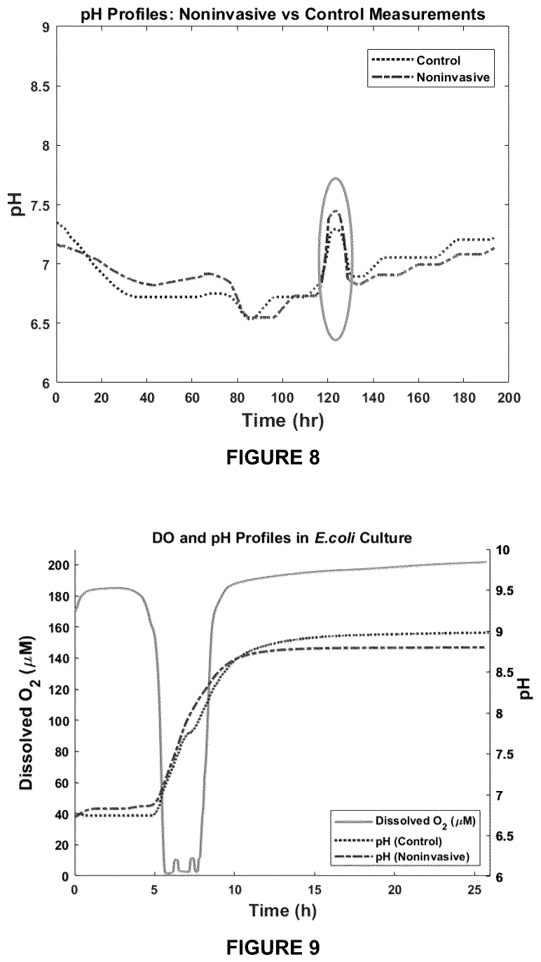

During 7 days of the CHO culture, the pH was monitored through control and noninvasive methods simultaneously. The pH profiles obtained from both techniques are shown in FIG. 8.

Seeding density was 2.85×10⁴ cells/cm² and final density reached 7.1×10⁴ cells/cm² indicating cell growth. Both pH profiles in FIG. 8 show a decrease in pH during the first part of the culture until time point of about 85 hours. This is potentially due to the lactate and CO₂ production during cell metabolism. An interesting event during the second part of the process, marked in FIG. 8, was the unintentional disconnection of the CO₂ supply to the incubator in the time range of 115 hours to 123 hours. This led to a decline in the CO₂ level inside the incubator which resulted in a decrease in the dissolved CO₂ level and an increase in the pH level in the cell culture medium. After the CO₂ supply was reconnected to the incubator, a gradual decrease in pH level was observed in the time range of 123 hours to 133 hours. This observation highlights the influence of CO₂ on the pH in the cell culture medium and the efficacy of the noninvasive method in tracking pH changes in the cell culture medium. Furthermore, the Pearson correlation between the noninvasive and control pH measurements is 90% which confirms the efficacy of the noninvasive technique.

E. Noninvasive pH Measurement in E. coli Culture Process

The E. coli culture process started with an initial OD of 0.9 and reached 6.12 after 25 hours, indicating cell growth. FIG. 9 shows the pH profiles obtained through noninvasive and control methods.

In the initial phase of the culture, minimal change in pH profile is observed, which could be due to low cell metabolism. In the second part of the culture, between 5 to 10 hours of the culture, an increase in pH profiles is observed, which is concomitant with a decrease in DO profile. This likely potentially due to the cell growth and production of alkaline products. When a protein-rich complex media is used, the cells cleave off ammonia from the contained amino acids as they have a much greater demand for the carbon. As a result, ammonium ions form in the aqueous solution and causes an increase in pH. After the time point of 10 hours, pH and DO profiles change in a smaller range. The Pearson correlation between the pH measurements from noninvasive and control methods is 98%, and this indicates that the noninvasive measurements are comparable with control measurements.

### F. Flow Cell Measurements

i. Sensor Evaluations with Medium

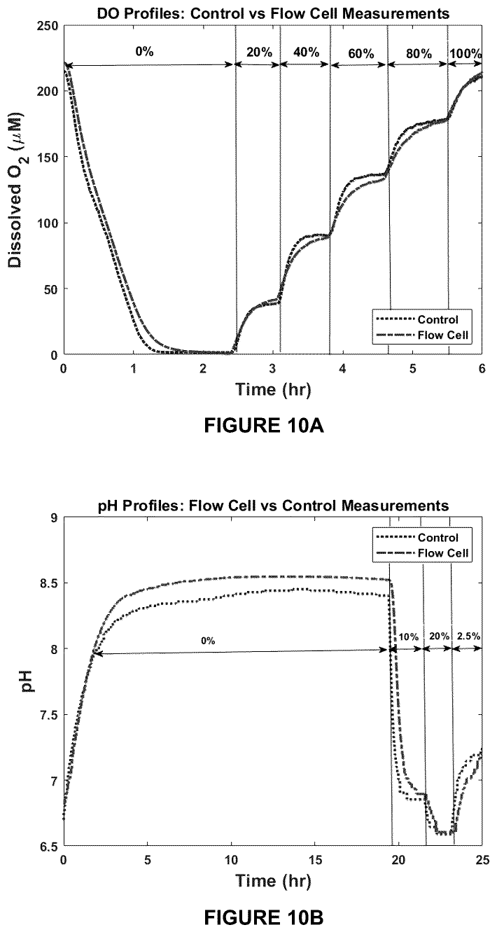

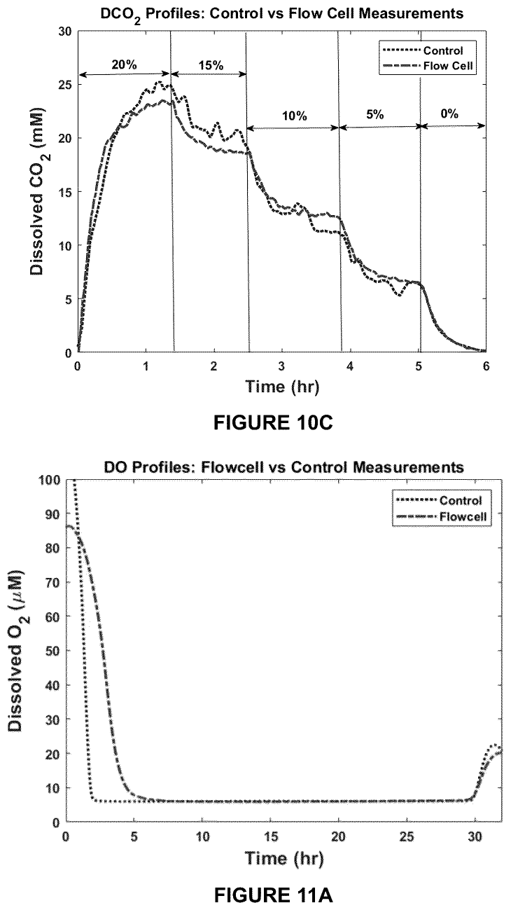

The online measurements of DO, pH, and DCO₂ were obtained through flow cell and compared with control measurements. The measurements from both techniques are presented in FIGS. 10A, 10B, and 10C, respectively. The percentages of gases sparged in the cell culture medium are shown for different periods of time in each figure.

Changing the percentage of the gases sparged in the medium results in changes in the concentration of gases dissolved in the medium and the pH of the medium. The figures show that the profiles obtained through the flow cell and the control profiles are comparable. Furthermore, the Pearson correlation between control and flow cell measurements for DO, pH, and DCO₂ are 99.57%, 98.27%, and 99.12%, respectively. This indicates that the flow cell is successful in tracking changes inside the cell culture vessel.

ii. E. coli Culture Process

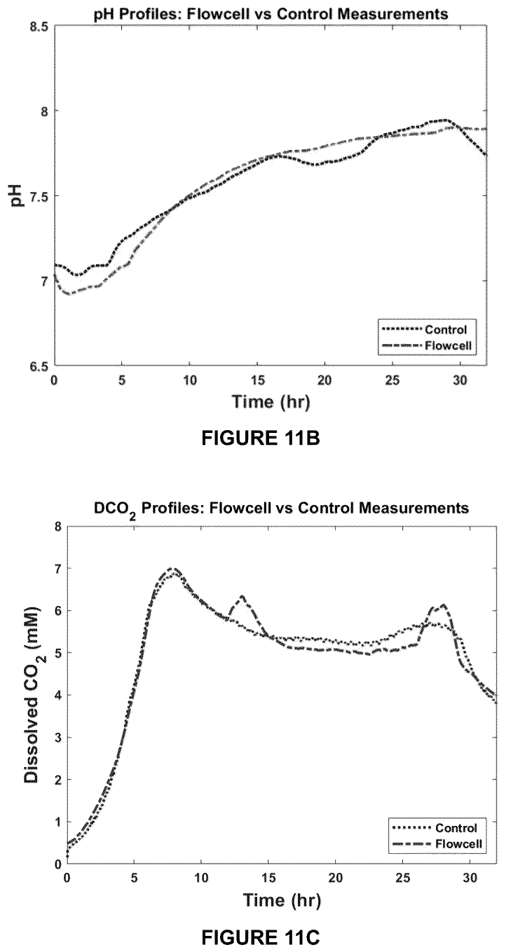

E. coli was cultured in a 2L shake flask, and the medium was continuously circulated between the shake flask and the flow cell. During the process, DO, pH, and DCO₂ were simultaneously measured through the control method inside the shake flask and the flow cell. FIGS. 11A, 11B, and 11C depict the DO, pH, and DCO₂ profiles, respectively. The Pearson correlations between control and flow cell measurements for DO, pH, and DCO₂ are 61%, 73%, and 99%, respectively, confirming the efficacy of the flow cell in tracking changes inside the cell culture vessel.

In FIG. 11A, the initial delay observed for DO profile is potentially the result of the formation of an air pocket within the DO sampler during the manufacturing process of the flow cell. This explanation seems true because the Pearson correlation obtained from the gas-sparge experiment described above using a different flow cell is very high (99.57%).

iii. Flow Cell Delay

Throughout the E. coli culture experiment described in the Methods and Materials section, the flow rate for transferring the sample to the flow cell was 0.25 ml/s, the inner diameter of the transfer tube was approximately 0.31 cm, and the length of the tube transferring the sample to the flow cell was approximately 183 cm. Therefore, the time required for transferring the sample to the flow cell is approximately 58 seconds calculated using Equation (1). By considering 27 ml as the total volume of the flow cell, the residence time is approximately 108s estimated using Equation (2). The thickness of silicone and cellulose membranes utilized in the flow cell was respectively 100 μm and 30 μm. Therefore, based on Equation (3), the approximate time for diffusion of protons through cellulose membrane, diffusion of oxygen through silicone membrane, and diffusion of carbon dioxide through silicone membrane are, respectively, 16.7 seconds, 1.5 seconds, and 2.3 seconds. The diffusion coefficient of 2.7×10-7 cm² sec-1 for protons through cellulose membrane, 3.25×10-5 cm² sec-1, for oxygen through silicone membrane, and 2.2×10-5 cm² sec-1 for carbon dioxide through silicone were obtained from literature and utilized in calculations (Fan et al. 2017; Markov et al. 2014; Yang and Kao. 2014).

### 5. Conclusions

Although online monitoring of dissolved O₂, pH, and dissolved CO₂ is critical in bioprocesses, nearly all existing technologies require some level of direct contact with the cell culture environment, posing a risk of contamination. The noninvasive monitoring system and method described herein enables online monitoring of DO, pH, and DCO₂ and can provide accurate results comparable to traditional invasive methods. As there is no direct contact with the cell culture medium, the noninvasive system and method eliminates the risk of contamination. This feature is especially crucial in cell therapy manufacturing processes, where the cells cannot be sterilized in the final stage. The noninvasive system and method described herein also addresses the concern regarding the cytotoxicity of sensing patches, which are directly placed in the media in traditional methods. Advantageously, the design of the noninvasive setup permits the replacement of malfunctioning parts of the monitoring system without interrupting the cell culture process. This makes it an appropriate monitoring system for long-term processes. Unlike currently available sensors, the application of the flow cell is not limited to specific cell culture processes, and has the potential to be used in different cell culture processes with different volumes.

### EXAMPLE 2

An area in the T-flask bottom wall was modified as shown in FIG. 2C, wherein a circular hole was drilled and a gas-permeable silicone film was attached to the hole. The hole with the gas-permeable silicone film was mechanically sealed to create a sterile environment in the T-flask. A sampler, as described herein (e.g., as illustrated in FIGS. 2A and 2B), was attached to the outside of the flask at the hole. The gases (O₂ and CO₂) diffuse through the permeable membrane into the sampler. A detection device, i.e., a sensor, was connected to the sampler by means of tubing. A micropump in the sensor brings any gases diffusing in the sampler to the sensor, and the concentration of 02 and CO₂ was measured.

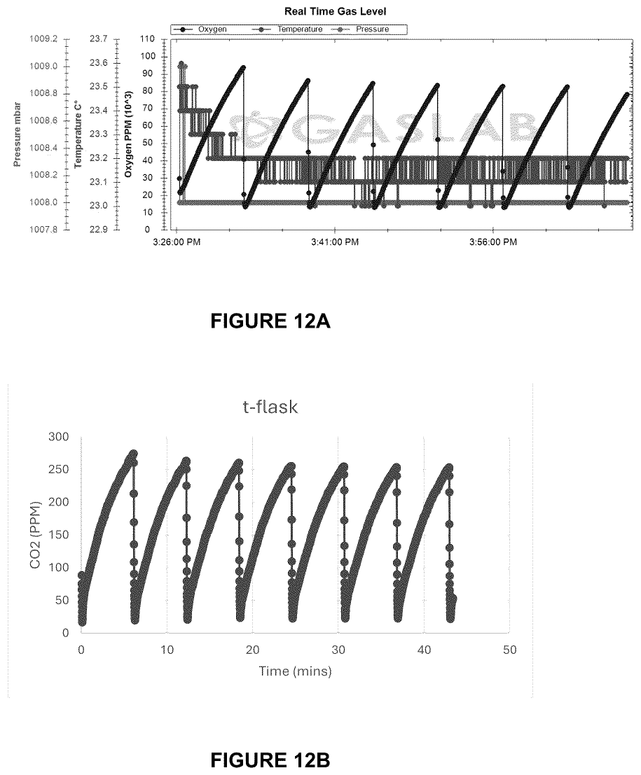

As shown in FIGS. 12A and 12B, the sampler communicatively connected to the sensor was able to measure both the concentration of 02 and CO₂ in the T-flask.

Although the invention has been variously disclosed herein with reference to illustrative embodiments and features, it will be appreciated that the embodiments and features described hereinabove are not intended to limit the invention, and that other variations, modifications and other embodiments will suggest themselves to those of ordinary skill in the art, based on the disclosure herein. The invention therefore is to be broadly construed, as encompassing all such variations, modifications and alternative embodiments within the spirit and scope of the claims hereafter set forth.

### REFERENCES

Abou-el-Enein M, Elsallab M, Feldman SA, Fesnak AD, Heslop HE, Marks P, Till BG, Bauer G, Savoldo B (2021) Scalable Manufacturing of CAR T Cells for Cancer Immunotherapy. Blood Cancer Discovery 2:408-422.
Calculator Academy (2024). Diffusion Time Calculator. https://calculator.academy/diffusion-time-calculator/. Accessed 17 Jun. 2024.
Chatterjee M, Ge X, Uplekar S, Kostov Y, Croucher L, Pilli M, Rao G (2015) A unique noninvasive approach to monitoring dissolved O₂ and CO₂ in cell culture: Noninvasive Approach to Monitoring O 2 and CO 2. Biotechnol Bioeng 112:104-110.
Fan, Z., Chen, J., Guo, W., Ma, F., Sun, S., & Zhou, Q. (2017). Crystallinity of regenerated cellulose from [Bmim] Cl dependent on the hydrogen bond acidity/basicity of anti-solvents. RSC advances, 7 (65), 41004-41010.
Ge, X., & Rao, G. (2012a). Real-time monitoring of shake flask fermentation and off gas using triple disposable noninvasive optical sensors. Biotechnology progress, 28 (3), 872-877.
Ge, X., Kostov, Y., Tolosa, L., & Rao, G. (2012b). Study on low-cost calibration-free pH sensing with disposable optical sensors. Analytica chimica acta, 734, 79-87.
Gupta, P. A., Ge, X., Kostov, Y., and Rao, G. (2013). A completely noninvasive method of dissolved oxygen monitoring in disposable small-scale cell culture vessels based on diffusion through permeable vessel walls. Biotechnol. Prog. 30 (1),172-177.
Kattipparambil Rajan D, Patrikoski M, Verho J, Sivula J, Ihalainen H, Miettinen S, Lekkala J (2016) Optical non-contact pH measurement in cell culture with sterilizable, modular parts. Talanta 161:755-761.
Klein, S. G., Alsolami, S. M., Steckbauer, A., Arossa, S., Parry, A. J., Ramos Mandujano, G., . . . & Duarte, C. M. (2021). A prevalent neglect of environmental control in mammalian cell culture calls for best practices. Nature Biomedical Engineering, 5 (8), 787-792.
Kroll P, Herwig C, Mueller H (2019) Control of dissolved carbon dioxide in driving force limited cell culture processes.
Lashkari, S. (2017). Controlling recombinant Escherichia coli cultures to the boundary of oxidative and overflow metabolism (BOOM) for robust efficient growth, ([Clemson (SC)]: Clemson University). [dissertation/master's thesis].
Lee, S., Ibey, B. L., Coté, G. L., & Pishko, M. V. (2008). Measurement of pH and dissolved oxygen within cell culture media using a hydrogel microarray sensor. Sensors and Actuators B: Chemical, 128 (2), 388-398.
Markov, D. A., Lillie, E. M., Garbett, S. P., & McCawley, L. J. (2014). Variation in diffusion of gases through PDMS due to plasma surface treatment and storage conditions. Biomedical microdevices, 16, 91-96.
Rahmatnejad V, Tolosa M, Ge X, Rao G (2022). A novel approach to noninvasive monitoring of dissolved carbon dioxide in small-scale cell culture processes. Front Bioeng Biotechnol 10:968294.
Rao, G. Sensor Techknowledge: Sensors for Process Development: A Practical Guide. UMBC Chemical, Biochemical & Environmental Engineering Department. 2020.
Sander, R. (2015). Compilation of Henry's law constants (version 4.0) for water as solvent. Atmos. Chem. Phys. 15 (8), 4399-4981.
Takahashi, M., Sawada, Y., and Aoyagi, H. (2017). Erratum to: Development of a circulation direct sampling and monitoring system for O₂ and CO₂ concentrations in the gas-liquid phases of shake-flask systems during microbial cell culture. Amb. Express 7 (1), 175.
Tolosa, L., Kostov, Y., Harms, P., & Rao, G. (2002). Noninvasive measurement of dissolved oxygen in shake flasks. Biotechnology and Bioengineering, 80 (5), 594-597.
Vallejos, J. R., Brorson, K. A., Moreira, A. R., & Rao, G. (2010). Dissolved oxygen and pH profile evolution after cryovial thaw and repeated cell passaging in a T-75 flask. Biotechnology and bioengineering, 105 (6), 1040-1047.
Yang, L. J., & Kao, A. F. (2010 January). Gas permeation in PDMS monitored by on-site pressure sensors. In 2010 IEEE 5th International Conference on Nano/Micro Engineered and Molecular Systems (pp. 352-355). IEEE.

1. A system for noninvasively detecting and quantitating species within a container, said system comprising:
the container, wherein the container comprises at least one hole or port in a wall of the container, wherein a membrane covers, or is positioned within, each hole or port, and wherein the membrane permits the passage of the species to be detected and quantitated therethrough;
at least one sensing device, wherein the at least one sensing device is positioned on, or in proximity to, the membrane, wherein the at least one sensing device is not in contact with any fluid present in the container, and wherein the at least one sensing device collects the species that pass through the membrane, reacts with the species that pass through the membrane, or both; and
a detection device for quantitating the species collected in, reacted with, or both collected in and reacted with, the at least one sensing device.

2. The system of claim 1, wherein the species are selected from protons, oxygen, carbon dioxide, glucose, glutamine, phosphate, peptides, proteins, lactate, acetate, ammonia, amino acids, oxidizing agents, reducing agents, ions, micronutrients, cytokines, and other diffusible soluble analytes.

3. (canceled)

4. The system of claim 1, wherein the container is a cell culture flask, a bioreactor or a flow cell.

5. (canceled)

6. The system of claim 1, wherein the container comprising the membrane is fluid-tight and sterile.

7.-8. (canceled)

9. The system of claim 1, wherein the at least one sensing device comprises an optical sensing patch, wherein the optical sensing patch is positioned between the membrane and an optically transparent layer.

10. (canceled)

11. The system of claim 9, wherein the optical sensing patch is positioned within a sampler, wherein the sampler comprises a gasket and the sampler can be positioned in proximity of the membrane such that species passing through the membrane are contained in the sampler.

12.-16. (canceled)

17. The system of claim 1, wherein the at least one sensing device comprises a sampler, wherein the sampler comprises a base, at least one borehole, and a gasket, wherein the sampler can be positioned over the membrane such that species passing through the membrane are contained in the sampler, and wherein the sampler can be communicatively connected to the detection device using tubing that is connected to the sampler via the at least one borehole.

18. The system of claim 1, wherein the at least one sensing device is an ion-selective electrode selected from pH, phosphate, magnesium, ammonium, calcium, sodium, potassium, carbonate, sulfate, and fluoride.

19. The system of claim 1, wherein the at least one sensing device comprises a flow channel engraved into a layer, wherein the flow channel is positioned in contact with the membrane so that the species can pass through the membrane into the flow channel and flows to a detection device for quantitation therein.

20.-22. (canceled)

23. A flow cell for noninvasively detecting and quantitating species, said flow cell comprising:
a layer for positioning of a first sensing device;
a first membrane layer, wherein the first membrane layer permits the passage of a species to be detected by the first sensing device;
a layer comprising a media channel;
a second membrane layer, wherein the second membrane layer permits the passage of a species to be detected by a second sensing device; and
a layer for positioning of the second sensing device,
wherein the layer comprising the media channel can be communicatively connected to a vessel comprising a medium, wherein the medium comprises species to be detected and quantitated.

24. The flow cell of claim 23, wherein the first and second sensing devices are positioned on, or in proximity to, the first and second membranes, respectively.

25.-26. (canceled)

27. The flow cell of claim 23, wherein the layer comprising a media channel comprises an inlet and an outlet that can be communicatively connected to a vessel comprising the medium.

28. The flow cell of claim 23, wherein at least one sensing device comprises an optical sensing patch.

29.-31. (canceled)

32. The flow cell of claim 23, wherein the at least one sensing device is an ion-selective electrode selected from pH, phosphate, magnesium, ammonium, calcium, sodium, potassium, carbonate, sulfate, and fluoride.

33. The flow cell of claim 23, wherein the at least one sensing device comprises a flow channel engraved into the layer, wherein the flow channel is positioned in contact with the respective membrane so that the species can pass through the membrane into the flow channel and flows to a detection device for quantitation therein.

34.-36. (canceled)

37. A system for noninvasively detecting and quantitating species in a medium contained in a vessel, said system comprising:
a flow cell of claim 23;
a pump; and
a vessel,
wherein the flow cell is communicatively connected to the vessel comprising the medium for detection and quantitation of a species contained therein.

38.-39. (canceled)

40. A system for noninvasively detecting and quantitating species in a medium contained in a vessel, said system comprising:
a flow cell;
a pump; and
a vessel,
wherein the flow cell is communicatively connected to the vessel comprising the medium for detection and quantitation of a species contained therein, and wherein the flow cell comprises:
a flow cell container, wherein the flow cell container comprises at least one hole or port in a wall of the flow cell container, an inlet and an outlet, wherein a membrane covers, or is positioned within, each hole or port, and wherein the membrane permits the passage of the species to be detected and quantitated therethrough;
at least one sensing device, wherein the at least one sensing device is positioned on, or in proximity to, the membrane, wherein the at least one sensing device collects the species that pass through the membrane, reacts with the species that pass through the membrane, or both; and
a detection device for quantitating the species collected in, reacted with, or both collected in and reacted with, the at least one sensing device.

41. The system of claim 40, wherein the species are selected from protons, oxygen, carbon dioxide, glucose, glutamine, phosphate, peptides, proteins, lactate, acetate, ammonia, amino acids, oxidizing agents, reducing agents, ions, micronutrients, cytokines, and other diffusible soluble analytes.

42. (canceled)

43. The system of claim 40, wherein the membrane is positioned within a ring, wherein the ring fits within the hole or port of the flow cell container.

44.-52. (canceled)

53. The system of claim 40, wherein the at least one sensing device comprises a sampler, wherein the sampler comprises a base, at least one borehole, and a gasket, wherein the sampler can be positioned over the membrane such that species passing through the membrane are contained in the sampler, and wherein the sampler can be communicatively connected to a detection device using tubing that is connected to the sampler via the at least one borehole.

54. The system of claim 40, wherein the at least one sensing device comprises a flow channel engraved into a layer, wherein the flow channel is positioned in contact with the membrane so that the species can pass through the membrane into the flow channel and flows to a detection device for quantitation therein.

55.-59. (canceled)
